(19) United States
(12) Patent Application Publication
(10) Pub. No.: US 2026/0317271 A1
(43) Pub. Date: Oct. 15, 2026

(54) CROSSLINKING AGENT, QUANTUM DOTS AND COMPOSITIONS THEREOF, QUANTUM DOT LIGHT EMITTING LAYER AND MANUFACTURING METHOD THEREOF, LIGHT EMITTING DEVICE AND DISPLAY DEVICE

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Bing FANG, Beijing (CN); Xu YUAN, Beijing (CN); Haoze YANG, Beijing (CN); Yichi ZHANG, Beijing (CN)

(21) Appl. No.: 18/996,629

(22) PCT Filed: Nov. 13, 2023
(86) PCT No.: PCT/CN2023/131198

§ 371 Date: Jan. 17, 2025

(51) Int. Cl. C07C 247/18; H10K 50/115; H10K 50/16; H10K 85/10

(52) U.S. Cl. CPC C07C 247/18; H10K 50/115; H10K 50/16; H10K 85/141

(57) ABSTRACT

The disclosure provides a crosslinking agent, quantum dots, a quantum dot composition, a quantum dot light emitting layer and a preparation method thereof, a light emitting device and a display device. The crosslinking agent has a general formula R—(R1)ₙ, in which R includes at least three reaction sites connected with R1, and R is selected from any one of following groups:

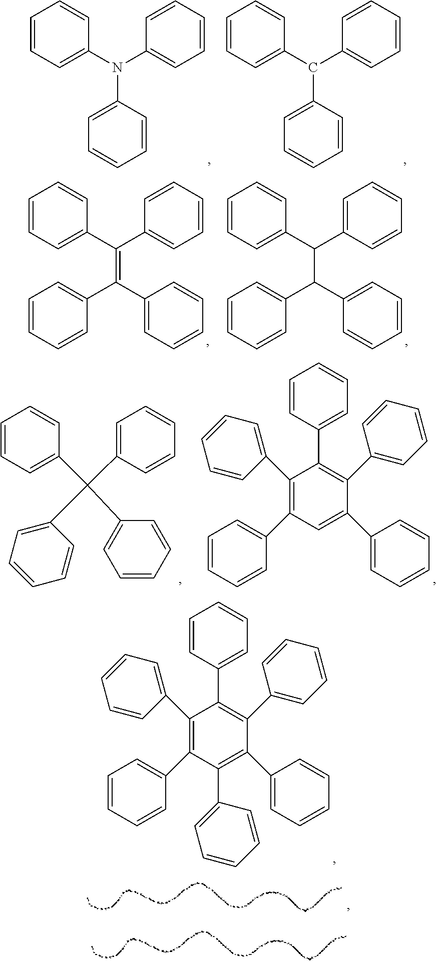

represents a polymer chain, and functional groups of the polymer chain includes at least one of carboxyl group, hydroxyl group and aldehyde group. R1 includes an azide group N₃, n is a positive integer greater than or equal to 3, and a value of n is equal to a number of the reaction sites.

### TECHNICAL FIELD

The present disclosure relates to the field of display technology, and in particular, to a crosslinking agent, quantum dots and compositions thereof, a quantum dot light emitting layer and manufacturing method thereof, a light emitting device and a display device.

### BACKGROUND

Light emitting Diode (LED) emits light by releasing energy through the recombination of electrons and holes. Light emitting diodes comprise, but are not limited to, organic light emitting diodes (OLED) and quantum dot light emitting diodes (QLED). Quantum dots are semiconductor nanomaterials that can bind excitons in three dimensions. Quantum dots have great application potential in high quality displays due to their excellent characteristics such as high quantum efficiency, narrow excitation spectrum, high photostability, long fluorescence lifetime and good solution processing compatibility. Quantum dot light emitting diode is a device using quantum dots as light emitting materials. Compared with organic light emitting diodes, it has outstanding advantages such as lower energy consumption, higher color purity and wider color gamut, therefore quantum dot light emitting technology has become the most potential next-generation self-luminous display technology.

### SUMMARY

According to an aspect of the present disclosure, a crosslinking agent is provided. The crosslinking agent has a general formula R—(R1)ₙ, wherein R comprises at least three reaction sites connected with R1, and R is selected from any one of following groups:

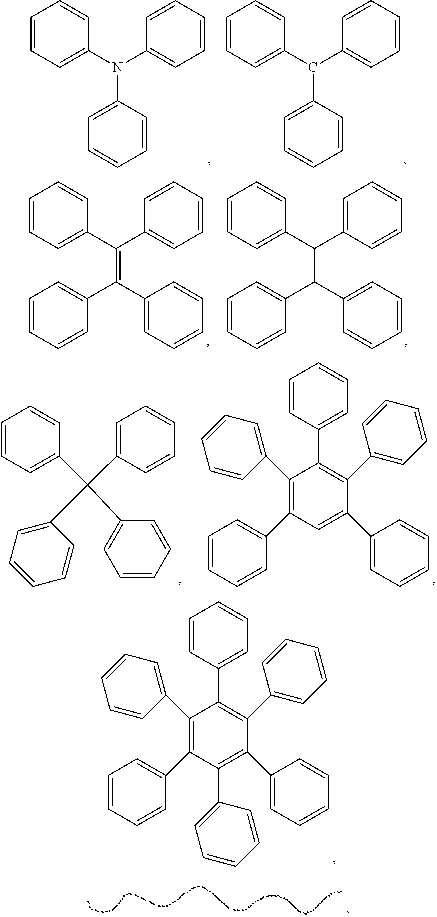

wherein

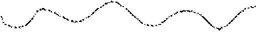

represents a polymer chain, and functional groups of the polymer chain comprise at least one of carboxyl group, hydroxyl group and aldehyde group, and wherein R1 comprises an azide group N₃, n is a positive integer greater than or equal to 3, and a value of n is equal to a number of the reaction sites.

In some embodiments, a chemical formula of R1 is

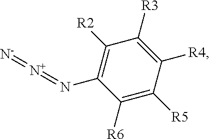

wherein any one of R2~R6 is a conjugated group or a non-conjugated group, and the conjugated group or the non-conjugated group is connected with the reaction site on R, and wherein remaining groups in R2~R6 except the group connected with the reaction site on R are same as or different from each other, and are respectively selected from fluorine atoms or hydrogen atoms.

In some embodiments, the non-conjugated group comprises at least one of ester group, amide group, ether group, imine group and carbon-nitrogen double bond.

In some embodiments, the conjugated group comprises a carbon-carbon double bond.

In some embodiments, the chemical formula of R1 is

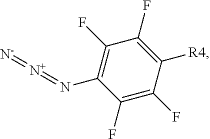

wherein R4 is connected with the reaction site on R.

In some embodiments, n is equal to 4, and a chemical formula of the crosslinking agent is

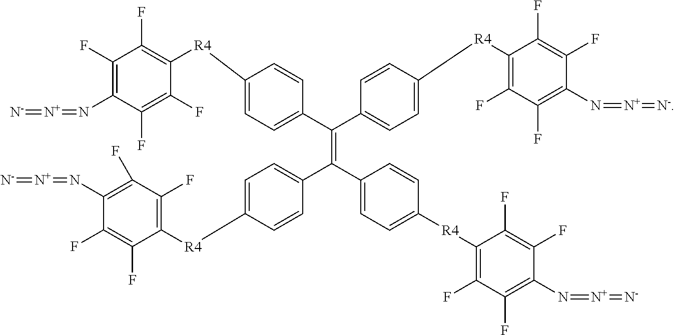

In some embodiments, the chemical formula of the crosslinking agent

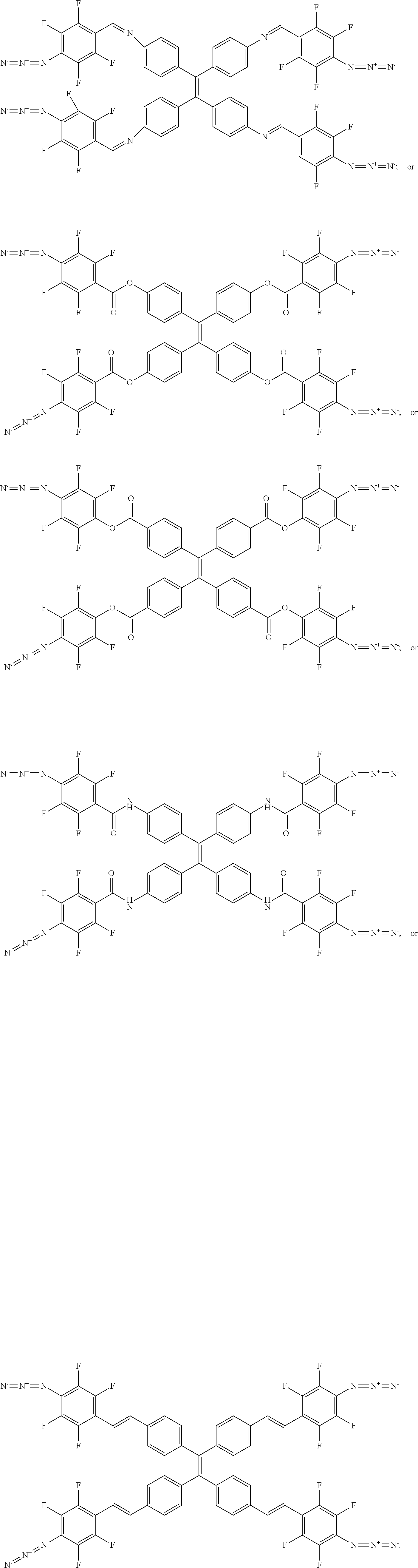

According to another aspect of the present disclosure, a quantum dot is provided. The quantum dot comprises a quantum dot body and a ligand coordinated on the quantum dot body, wherein the ligand comprises a carbon-hydrogen bond, and the ligand is configured to crosslink with the crosslinking agent described in any of the previous embodiments under light radiation.

In some embodiments, the ligand is selected from any one of oleylamine, isooctyl mercaptan, mercaptoacetic acid, mercaptopropionic acid, oleic acid and 2-mercaptoethyl Boc amine.

In some embodiments, the quantum dot is configured to emit red light or blue light, and the ligand is oleic acid.

In some embodiments, the quantum dot is configured to emit green light, and the ligand is 2-mercaptoethyl Boc amine.

According to yet another aspect of the present disclosure, a quantum dot composition is provided. The quantum dot composition comprises a mixed solution comprising quantum dots described in any of the previous embodiments, a crosslinking agent described in any of the previous embodiments, and a solvent. A concentration of the quantum dots in the mixed solution is about 20-35 mg/ml, and a concentration of the crosslinking agent in the mixed solution is about 0.1-2.0 mg/ml.

In some embodiments, the solvent is selected from any one of propylene glycol methyl ether acetate, toluene, chlorobenzene, octane, water and alcohol solvents.

According to another aspect of the present disclosure, a quantum dot light emitting layer is provided. The quantum dot light emitting layer comprises a plurality of quantum dots. At least some of the quantum dots comprise quantum dot bodies and ligands coordinated on the quantum dot bodies, and the quantum dot light emitting layer is generated by crosslinking quantum dots with the ligands and the crosslinking agent described in any of the previous embodiments.

In some embodiments, a chemical formula of the quantum dot light emitting layer is R—(R1′-R7-QD)n, wherein QD represents quantum dots, R7 represents the ligand and comprises carbon-hydrogen bonds, and R1′ represents a group in which the azide group N₃ of the group R1 is replaced by a group NH.

In some embodiments, the chemical formula of the quantum dot light emitting layer is

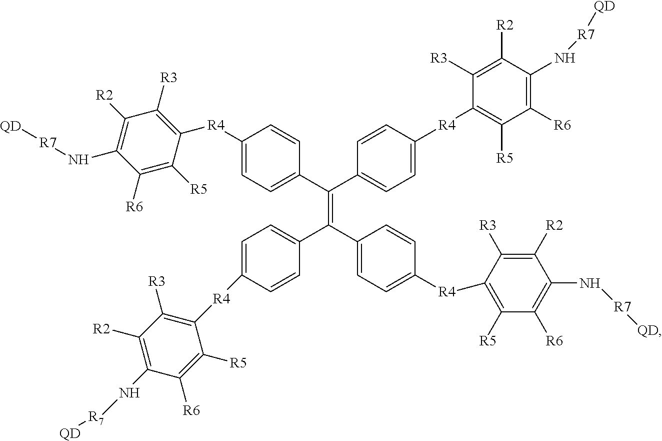

wherein R4 is a conjugated group or a non-conjugated group, and R2, R3, R5 and R6 are same or different from each other and are respectively selected from fluorine atoms or hydrogen atoms.

In some embodiments, the quantum dot light emitting layer is configured to emit red light or blue light, the chemical formula of the quantum dot light emitting layer is

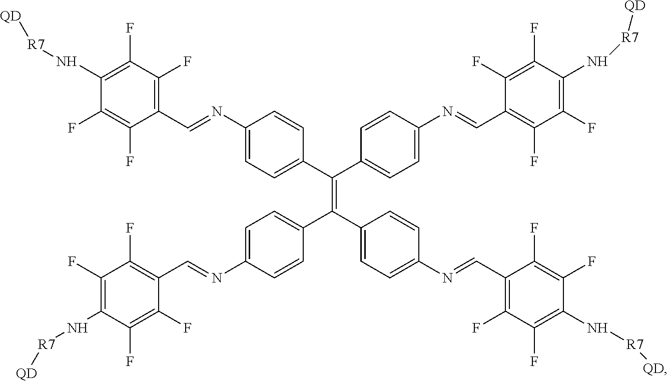

and R7 is oleic acid.

In some embodiments, the quantum dot light emitting layer is configured to emit green light, the chemical formula of the quantum dot light emitting layer is

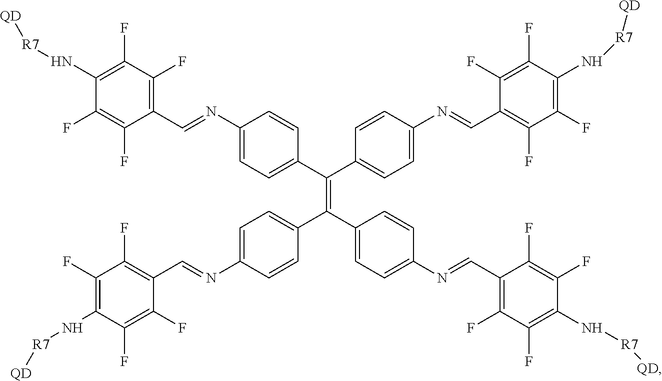

and R7 is 2-mercaptoethyl Boc amine.

In some embodiments, the quantum dot light emitting layer comprises a plurality of first quantum dot patterns, a plurality of second quantum dot patterns, a plurality of first sub-pixel regions, and a plurality of second sub-pixel regions, the first quantum dot patterns are configured to emit red light and the second quantum dot patterns are configured to emit green light, the first quantum dot patterns are only in the first sub-pixel regions but not in the second sub-pixel regions, and the second quantum dot patterns are only in the second sub-pixel regions but not in the first sub-pixel regions.

In some embodiments the quantum dot light emitting layer further comprises a plurality of third quantum dot patterns configured to emit blue light and a plurality of third sub-pixel regions, the first quantum dot patterns are only in the first sub-pixel regions but not in the second sub-pixel regions and the third sub-pixel regions, the second quantum dot pattern are only in the second sub-pixel regions but not in the first sub-pixel regions and the third sub-pixel region, and the third quantum dot patterns are only in the third sub-pixel regions but not in the first sub-pixel regions and the second sub-pixel regions.

According to still another aspect of the present disclosure, a light emitting device is provided. The light emitting device a first electrode layer; an electron transport layer on the first electrode layer; the quantum dot light emitting layer described in any of the previous embodiments on a side of the electron transport layer away from the first electrode layer; a hole transport layer on a side of the quantum dot light emitting layer away from the first electrode layer; and a second electrode layer on a side of the hole transport layer away from the first electrode layer.

In some embodiments, the electron transport layer is nanoparticles.

According to yet another aspect of the present disclosure, a display device is provided. The device comprises a plurality of light emitting devices described in any of the previous embodiments, and at least two of the plurality of light emitting devices are configured to emit light of different colors.

According to another aspect of the present disclosure, a method for preparing a patterned quantum dot light emitting layer is provided. The method comprises: providing a substrate;
applying a mixed solution on the substrate, wherein the mixed solution comprises quantum dots and a crosslinking agent, wherein the quantum dots comprise quantum dot bodies and ligands coordinated on the quantum dot bodies, the ligands comprise carbon-hydrogen bonds, and the crosslinking agent is the crosslinking agent described in any of the previous embodiments; curing the mixed solution to form a cured layer; and exposing and developing the cured layer to form a patterned quantum dot light emitting layer.

In some embodiments, exposing and developing the cured layer comprises: exposing the cured layer by using a mask plate, allowing ultraviolet light to penetrate through the mask plate to expose the cured layer, and performing crosslinking reaction between the crosslinking agent and ligands on the surface of the quantum dots under ultraviolet light irradiation; and developing a crosslinked cured layer by using a developing solution, wherein a part of the cured layer in the non-exposed area is dissolved by the developing solution, and a part of the cured layer in the exposed area is not dissolved by the developing solution, so as to form the patterned quantum dot light emitting layer.

### BRIEF DESCRIPTION OF THE DRAWINGS

In the following, exemplary embodiments of the present disclosure will be described in detail with reference to the accompanying drawings, in which:

FIG. 1 shows a schematic diagram of the patterning principle of a quantum dot light emitting layer in the related art;

FIG. 2 shows an electrospectrogram of the quantum dot light emitting layer of FIG. 1;

FIG. 3 shows a schematic diagram of the patterning principle of another quantum dot light emitting layer in the related art;

FIG. 4 shows a photograph of the quantum dot light emitting layer of FIG. 3 after being patterned;

FIG. 5 shows some chemical formulas of a group R according to an embodiment of the present disclosure;

FIG. 6 shows a general formula of a group R1 according to an embodiment of the present disclosure;

FIG. 7 shows a general formula of a crosslinking agent according to an embodiment of the present disclosure;

FIG. 8 shows a general formula of another crosslinking agent according to an embodiment of the present disclosure;

FIG. 9 shows a general formula of yet another crosslinking agent according to an embodiment of the present disclosure;

FIG. 10 shows a general formula of yet another crosslinking agent according to an embodiment of the present disclosure;

FIG. 11 shows a general formula of yet another crosslinking agent according to an embodiment of the present disclosure;

FIG. 12 shows a general formula of yet another crosslinking agent according to an embodiment of the present disclosure;

FIG. 13 shows a general formula of yet another crosslinking agent according to an embodiment of the present disclosure;

FIG. 14 shows a chemical formula of a crosslinking agent according to an embodiment of the present disclosure;

FIG. 15 shows a chemical formula of a crosslinking agent according to an embodiment of the present disclosure;

FIG. 16 shows a synthesis route of the crosslinking agent of FIG. 15;

FIG. 17 shows the nuclear magnetic resonance hydrogen spectrum of the crosslinking agent of FIG. 15;

FIG. 18 shows a chemical formula of another crosslinking agent according to an embodiment of the present disclosure;

FIG. 19 shows a synthesis route of the crosslinking agent of FIG. 18;

FIG. 20 shows a chemical formula of yet another crosslinking agent according to an embodiment of the present disclosure;

FIG. 21 shows a synthesis route of the crosslinking agent of FIG. 20;

FIG. 22 shows a chemical formula of yet another crosslinking agent according to an embodiment of the present disclosure;

FIG. 23 shows a synthesis route of the crosslinking agent of FIG. 22;

FIG. 24 shows a chemical formula of yet another crosslinking agent according to an embodiment of the present disclosure;

FIG. 25 shows a synthesis route of the crosslinking agent of FIG. 24;

FIG. 26 shows a schematic structural diagram of a quantum dot according to an embodiment of the present disclosure;

FIG. 27 shows a general formula of a quantum dot light emitting layer after a crosslinking reaction between a crosslinking agent and quantum dots according to an embodiment of the present disclosure;

FIG. 28 shows a chemical formula of a quantum dot light emitting layer after a crosslinking reaction between a crosslinking agent and quantum dots according to an embodiment of the present disclosure;

FIG. 29 shows a synthesis route of the quantum dot light emitting layer of FIG. 28;

FIG. 30 shows the light emitting patterns of the quantum dot light emitting layer of FIG. 28 under different development conditions;

FIG. 31 shows the electrospectrograms of the quantum dot light emitting layer of FIG. 28 under different development conditions;

FIG. 32 shows a photograph of a green quantum dot light emitting layer after being patterned;

FIG. 33 shows a chemical formula of a quantum dot light emitting layer after a crosslinking reaction between a crosslinking agent and quantum dots according to an embodiment of the present disclosure;

FIG. 34 shows a synthesis route of the quantum dot light emitting layer of FIG. 33;

FIG. 35 shows light emitting patterns of green quantum dot light emitting layers with different concentrations according to an embodiment of the present disclosure;

FIG. 36 shows an electrospectrogram of a green quantum dot light emitting layer according to an embodiment of the present disclosure;

FIG. 37 shows light emitting patterns of blue quantum dots under different development conditions according to an embodiment of the present disclosure;

FIG. 38 shows light emitting patterns of a red quantum dot light emitting layer and a red-green quantum dot light emitting layer according to an embodiment of the present disclosure;

FIG. 39 shows an electrospectrogram and a light emitting pattern of a red quantum dot light emitting layer according to an embodiment of the present disclosure;

FIG. 40 shows an electrospectrogram and a light emitting pattern of a green quantum dot light emitting layer according to an embodiment of the present disclosure;

FIG. 41 shows a photograph of a patterned quantum dot light emitting layer obtained by using a crosslinking agent comprising a bisazide group in the related art;

FIG. 42 shows a schematic structural diagram of a light emitting device according to an embodiment of the present disclosure;

FIG. 43 shows a block diagram of a display device according to an embodiment of the present disclosure; and

FIG. 44 shows a flowchart of preparing a patterned quantum dot light emitting layer according to an embodiment of the present disclosure.

It should be understood that the drawings are only schematic illustrations of exemplary embodiments of the present disclosure, which are not limitations of the present disclosure and need not be drawn to scale. In addition, in the drawings, the same or similar parts are indicated by the same or similar reference numbers.

### DETAILED DESCRIPTION OF THE DISCLOSURE

In the following, the technical solutions in the embodiments of the disclosure will be described clearly and completely in connection with the drawings in the embodiments of the disclosure. Obviously, the described embodiments are only part of the embodiments of the disclosure, and not all of the embodiments. Based on the embodiments in the present disclosure, all other embodiments obtained by those of ordinary skills in the art under the premise of not paying out creative work pertain to the protection scope of the disclosure.

As used herein, unless a specific definition is provided otherwise, when a chemical bond is not drawn where it should be given, a hydrogen atom bond is bonded at the position.

Quantum dot light emitting diode display is a new display technology developed on the basis of organic light emitting diode display. A quantum dot light emitting diode display uses quantum dots as the light emitting layer, and its light emitting principle is that holes and electrons are injected into the quantum dot light emitting layer through the hole transport layer and the electron transport layer respectively, and the holes and electrons recombine in the quantum dot light emitting layer, thus realizing light emission. Compared with organic light emitting diode display, quantum dot light emitting diode display has the advantages of higher color saturation, wider color gamut, narrower emission peak and better stability. With the in-depth development of quantum dot technology, the research based on quantum dot display is deepening progressively, and with the continuous improvement of quantum efficiency, the level of industrialization can basically be achieved. Therefore, aiming at the preparation of quantum dot light emitting layer, it has become a future trend to develop new processes and technologies to make the preparation process of quantum dot light emitting diode display more excellent.

The traditional method of patterning quantum dot light emitting layer is to expose and develop the photoresist layer to obtain patterned photoresist. Then, the patterned photoresist is used to block the quantum dot light emitting layer and etch away the unnecessary parts, thus forming the patterned quantum dot light emitting layer. Although the traditional photoresist method can realize the patterning of quantum dots, the further application of this traditional method is limited due to the complicated process flow and poor solvent compatibility. To solve this limitation, it is urgent to develop new quantum dot patterning methods.

Recently, the industry has proposed that the patterned quantum dot light emitting layer can be obtained by direct exposure and development of quantum dot thin films without using photoresist. Such a manufacturing method is simple and low in cost, and a patterned quantum dot light emitting layer with high quality can be obtained, thereby realizing a quantum dot electroluminescent device with higher resolution. When the quantum dot light emitting layer is directly photoetched to realize patterning, it is necessary to add a crosslinking agent into the quantum dot solution, so that the crosslinking agent reacts with the ligand on the surface of the quantum dot to form a patterned quantum dot light emitting layer.

When a full-color device is prepared by patterning the quantum dot light emitting layer by direct method, after exposure and development, ideally, only red quantum dots exist in a red sub-pixel region without green and blue quantum dots, only green quantum dots exist in a green sub-pixel region without red and blue quantum dots, and only blue quantum dots exist in a blue sub-pixel region without red and green quantum dots. However, the reality is that some quantum dots often remain in sub-pixel regions that should not appear. For example, red quantum dots remain in green sub-pixel regions and blue sub-pixel regions in addition to red sub-pixel regions, which leads to that in the subsequent preparation of the green quantum dot light emitting layer and the blue quantum dot light emitting layer, the green sub-pixel region comprises not only the green quantum dots but also the residual red quantum dots, and the blue sub-pixel region comprises not only the blue quantum dots but also the residual red quantum dots, thus causing the color mixing problem and reducing the color gamut of the quantum dot electroluminescent device.

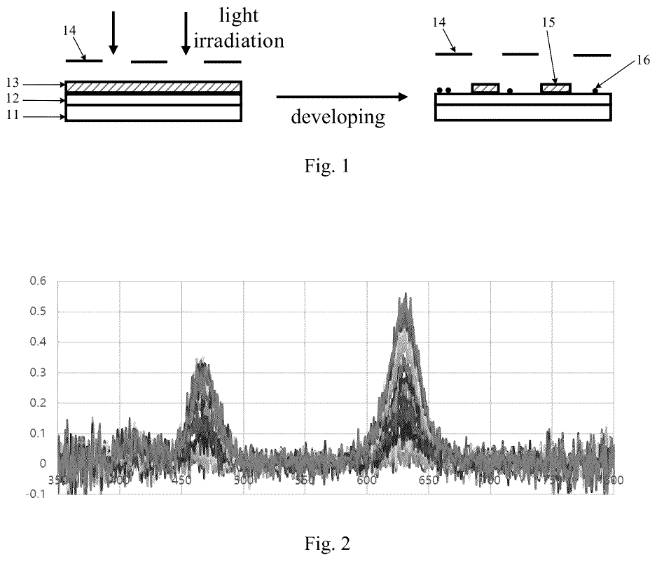

For example, FIG. 1 shows a schematic diagram of the patterning principle of a quantum dot light emitting layer in the related art. As shown in FIG. 1, an electron transport layer 12 is formed on a first electrode 11, and then a mixed solution (comprising red quantum dots, photosensitizer PAC, photoacid generator PAG, etc.) is spin-coated on the electron transport layer 12 and cured to form a quantum dot film layer 13. Ultraviolet light irradiates the quantum dot film layer 13 through the opening of the mask plate 14 to expose the quantum dot film layer 13, and then the quantum dot film layer 13 is developed by a developing solution. As a result, not only the patterned red quantum dot light emitting layer 15 is formed in the exposed area (red sub-pixel region), but also red quantum dots 16 remain in the unexposed area (e.g. green sub-pixel region and/or blue sub-pixel region). It is found that the reason why the red quantum dots 16 remain is that the part of the quantum dot film layer 13 located in the exposed area does not dissolve in the developing solution when developing with the developing solution, so that the patterned red quantum dot light emitting layer 15 can be formed. However, the electron transport layer 12 is formed by sol-gel method, there is a certain van der Waals force between the electron transport layer 12 and the quantum dots, and this van der Waals force causes the part of the quantum dot film layer 13 in the unexposed area to remain on the electron transport layer 12, thus causing the red quantum dots 16 to remain in the green sub-pixel region and/or blue sub-pixel region.

According to the above method, a patterned green quantum dot light emitting layer and a patterned blue quantum dot light emitting layer are respectively formed in the green sub-pixel regions and the blue sub-pixel regions, and then a hole transport layer and a second electrode are sequentially formed on a side of the red, green and blue quantum dot light emitting layer away from the electron transport layer 12 to form a quantum dot electroluminescent device.

As shown in FIG. 2, for example, when the green sub-pixel of the above-mentioned quantum dot electroluminescent device is tested by electrochemistry, with the gradual increase of voltage, it is found that the green sub-pixel not only has the electroluminescent peak of the green quantum dot, but also obviously has the electroluminescent peak of the red quantum dot, which indicates that the red quantum dot remains in the green sub-pixel region. Therefore, when preparing full-color devices, red quantum dots will remain in the green and/or blue sub-pixel regions, resulting in color mixing, which greatly reduces the color gamut range of quantum dot electroluminescent devices and weakens the advantages of quantum dots as electroluminescent materials.

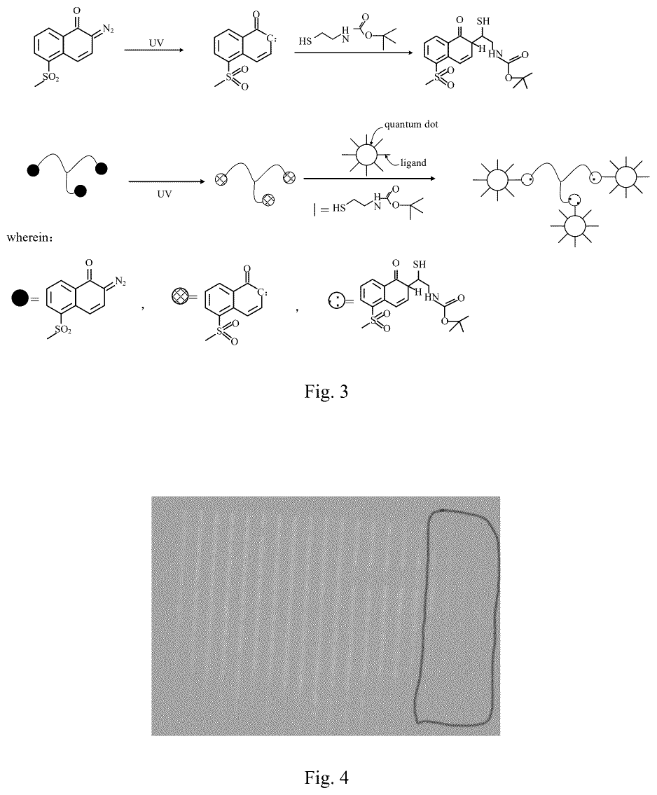

In order to solve the problem of quantum dot residue, the industry has developed crosslinking agents such as diazonaphthoquinone. As shown in FIG. 3, under ultraviolet irradiation, diazonaphthoquinone crosslinking agent

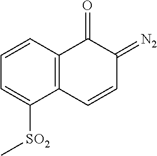

changes to

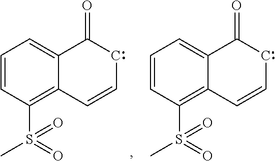

undergo crosslinking reaction with ligands on the surface of quantum dots to form a quantum dot light emitting layer. However, when developing the quantum dot light emitting layer with developing solution, it is found that although the number of residual quantum dots in unexposed areas is reduced, the quantum dot film layer in exposed areas will be washed away more or less by developing solution, so that the expected pattern shape cannot be formed, and the degree to which the quantum dot film layer in exposed areas is washed away fluctuates greatly with experiments, which is difficult to control.

FIG. 4 shows the quantum dot film layer in the exposed area. As shown in FIG. 4, the quantum dot pattern in the exposed area is damaged to varying degrees by the developing solution, especially the part circled by the solid black line, and the quantum dot pattern has been basically washed away by the developing solution, which seriously damages the pattern shape. The reason for this phenomenon may be that the degree of crosslinking between quantum dots and crosslinking agent in the exposed area is not enough, and the photoinitiation efficiency of crosslinking agent is low.

In order to solve the problems existing in the prior art, it is urgent to develop new crosslinking agents to enhance the stability of quantum dot light emitting layer patterns. The quantum dot light emitting layer formed by the crosslinking reaction between the new crosslinking agent and the quantum dots will not be damaged in the exposed area under the action of the developing solution, and the expected pattern shape can be well formed. At the same time, the part of the quantum dot light emitting layer in the unexposed area can be washed away by the developing solution without residue, thus avoiding the color mixing phenomenon caused by the residue of quantum dots.

Embodiments of the present disclosure provide a class of crosslinking agents having the general formula R—(R1)ₙ, wherein R comprises at least three reaction sites connected with R1.

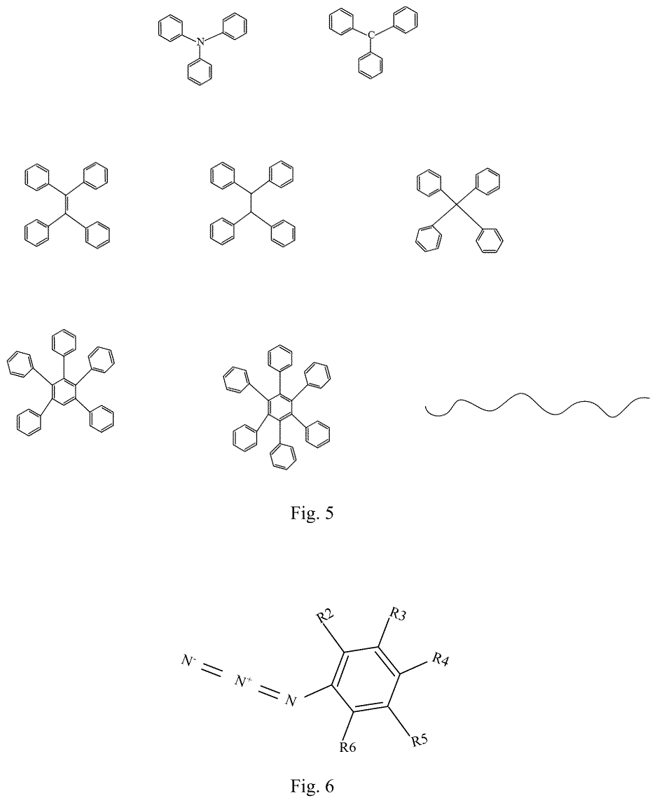

FIG. 5 shows the chemical formula of group R or mother nucleus R), which may be

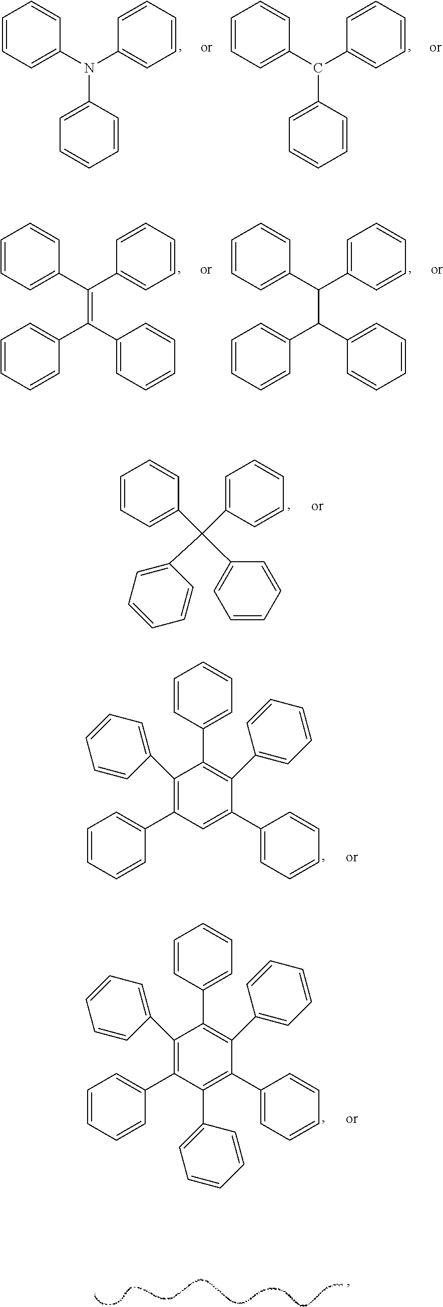

or other suitable groups.

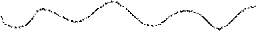

represents a polymer chain, and the functional group of the polymer chain comprises at least one of carboxyl group, hydroxyl group and aldehyde group. In the general formula R—(R1)ₙ, R1 comprises azide group N₃, n is a positive integer greater than or equal to 3, and the value of n is equal to the number of reaction sites on R.

Azide group N₃ has higher photoinitiation efficiency, lower exposure dose and better quantum dot patterning effect, so it is the preferred material as a crosslinking agent. The crosslinking agent provided by the embodiments of the present disclosure has the general formula R—(R1)ₙ, and the group R has at least three reaction sites that can be connected with the group R1, so that each group R may be connected with at least three groups R1, and each R1 comprises at least one azide group N₃, so that each crosslinking agent molecule comprises at least three azide groups N₃, which has a high degree of crosslinking. The quantum dot light emitting layer formed by the crosslinking reaction between the crosslinking agent and quantum dots has high stability, thus improving the stability of the quantum dot light emitting layer in the exposed area. Under the action of the developing solution, the part of the quantum dot light emitting layer in the exposure area will not be damaged by the developing solution, and the expected pattern shape can be well formed. At the same time, the part of the quantum dot light emitting layer in the unexposed area can be washed away by the developing solution without residue, thus avoiding the color mixing phenomenon caused by the residue of quantum dots. By using the crosslinking agent provided by the embodiments of the present disclosure, the problem of quantum dot residue can be solved, and high-quality quantum dot patterns can be obtained.

FIG. 6 shows the chemical formula of the group R1 in the general formula R—(R1)ₙ, which is

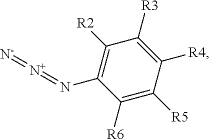

wherein any one of R2~R6 is a conjugated group or a non-conjugated group, and the conjugated group or the non-conjugated group is connected with the reaction site on R, and wherein remaining groups in R2~R6 except the group connected with the reaction site on R are same as or different from each other, and are respectively selected from fluorine atoms or hydrogen atoms. Depending on the position of the absorption peak, the chemical bond between R and R1 is designed as a conjugated group or a non-conjugated group. In some embodiments, the para group R4 of the azide group N3 is connected with the reaction site on R. In this case, the group R4 is a conjugated group or a non-conjugated group, and the remaining groups R2, R3, R5 and R6 are the same or different from each other and may be fluorine atoms or hydrogen atoms respectively. In some alternative embodiments, the ortho group R2 (or R6) of the azide group N₃ may be connected with the reaction site on R. In this case, the group R2 (or R6) is a conjugated group or a non-conjugated group, and the remaining groups R3, R4, R5, R6 (or R2, R3, R4, R5) are the same or different from each other and may be fluorine atoms or hydrogen atoms respectively. In some alternative embodiments, the meta group R3 (or R5) of the azide group N₃ may be connected with the reaction site on R. In this case, the group R3 (or R5) is a conjugated group or a non-conjugated group, and the remaining groups R2, R4, R5, R6 (or R2, R3, R4, R6) are the same or different from each other and may be fluorine atoms or hydrogen atoms respectively.

Azide group N₃ can generate nitrogen carbene radical under light irradiation, which has high activity and can undergo insertion reaction with carbon-hydrogen bonds of quantum dot ligands to form a crosslinked structure. When the para-position, ortho-position or meta-position of the azide group N₃ in the chemical formula

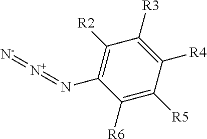

is fluorine atom, it is beneficial to better stabilize the nitrogen carbene radical and promote the insertion reaction between the crosslinking agent and the carbon-hydrogen bonds of the quantum dot ligands.

When the crosslinking agent absorbs at 254 nm, the group on R1 connected with the reaction site on R needs to be a non-conjugated group. Illustratively, the non-conjugated group may include at least one of an ester group, an amide group, an ether group, an imine group, and a carbon-nitrogen double bond.

When the crosslinking agent absorbs at 365 nm, the group on R1 connected with the reaction site on R needs to be a conjugated group. Illustratively, the conjugated group may include a carbon-carbon double bond. 365 nm is an ideal absorption peak position, because the absorption peak has little damage to quantum dots.

For the group on R1 connected with the reaction site on R, it is necessary to consider the principle of similar miscibility between molecules to realize the compatibility between the crosslinking agent and quantum dots. For example, if the quantum dot and the corresponding solvent have been determined, the compatibility of the crosslinking agent with the quantum dot can be achieved by designing the group type on R1 connected with the reaction site on R. For example, quantum dots have good dispersibility in solvent propylene glycol methyl ether acetate (PGMEA for short). When the solvent of quantum dots is PGMEA, because PGMEA contains ester groups, in order to improve the solvent compatibility between crosslinking agent and quantum dots, the group on R1 connected with the reaction site on R can also be selected as ester group.

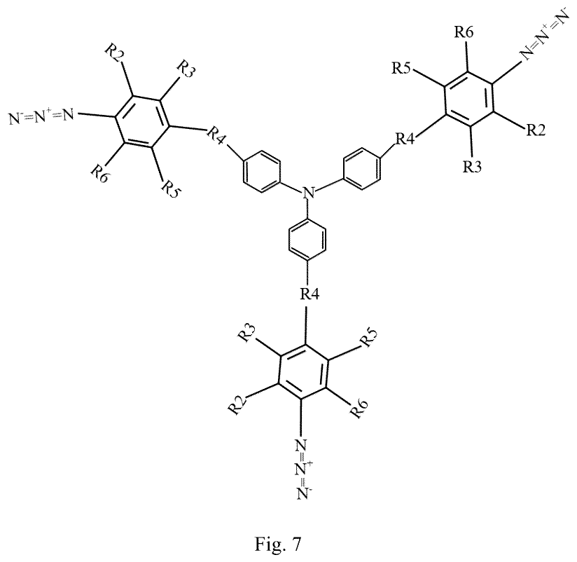

As shown in FIG. 7, when R is

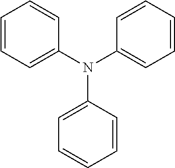 [SMILES: c1ccc(N(c2ccccc2)c2ccccc2)cc1]

and the group R4 of R1 is connected with the reaction site of R, the chemical formula of the crosslinking agent can be expressed as

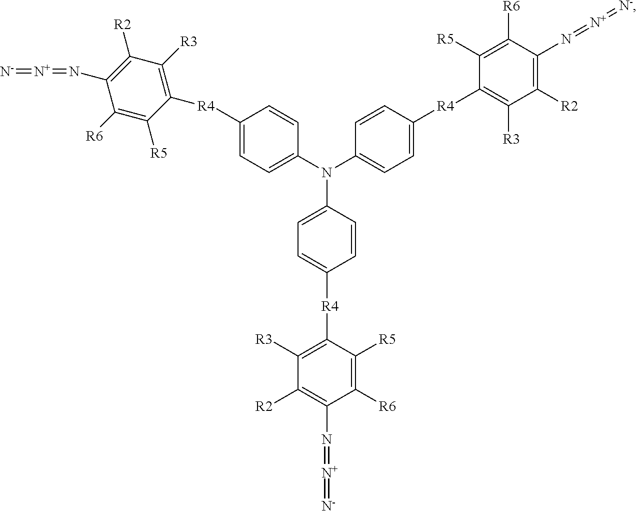

and the crosslinking agent includes three azide groups N3.

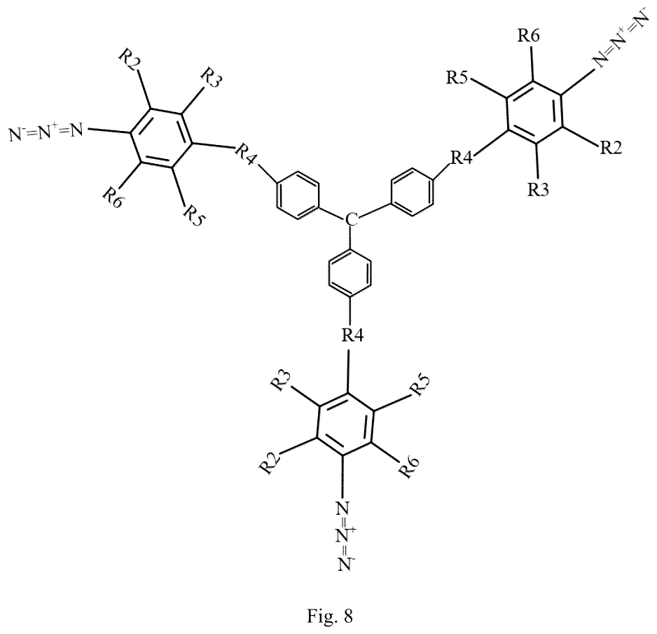

As shown in FIG. 8, when R is

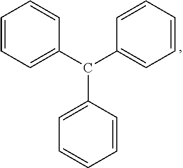 [SMILES: c1ccc(C(c2ccccc2)c2ccccc2)cc1]

and the group R4 of R1 is connected with the reaction site of R, the chemical formula of the crosslinking agent can be expressed as

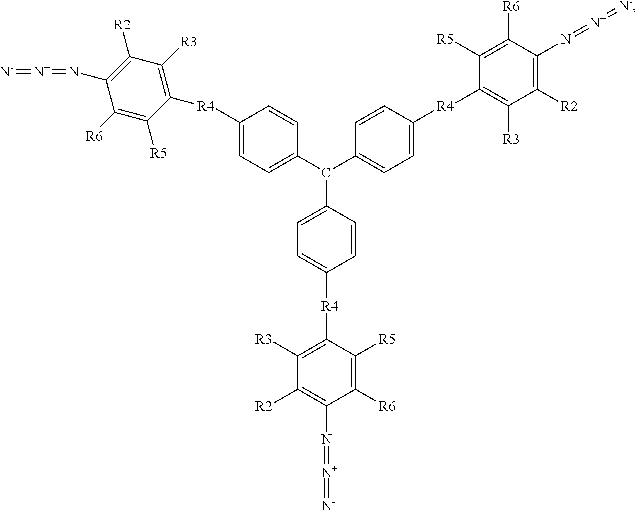

and the crosslinking agent includes three azide groups N3.

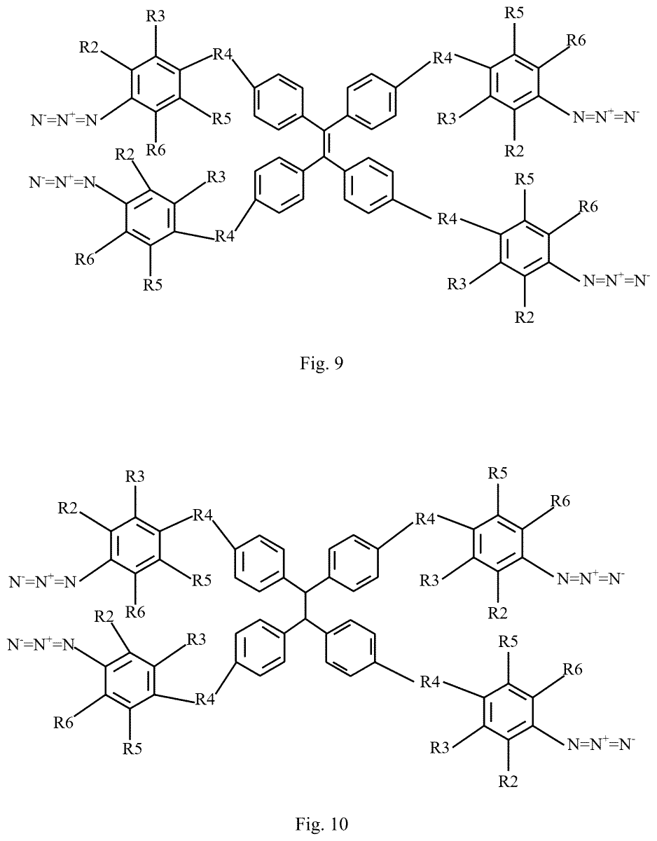

As shown in FIG. 9, when R is

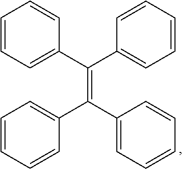 [SMILES: c1ccc(C(=C(c2ccccc2)c2ccccc2)c2ccccc2)cc1]

and the group R4 of R1 is connected with the reaction site of R, the chemical formula of the crosslinking agent can be expressed as

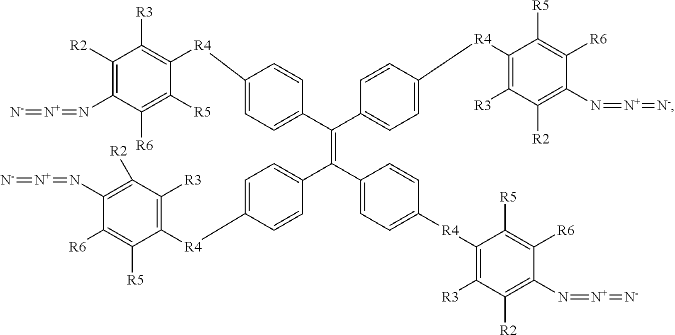

and the crosslinking agent includes four azide groups N3.

As shown in FIG. 10, when R

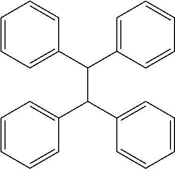 [SMILES: c1ccc(C(c2ccccc2)C(c2ccccc2)c2ccccc2)cc1]

is and the group R4 of R1 is connected with the reaction site of R, the chemical formula of the crosslinking agent can be expressed as

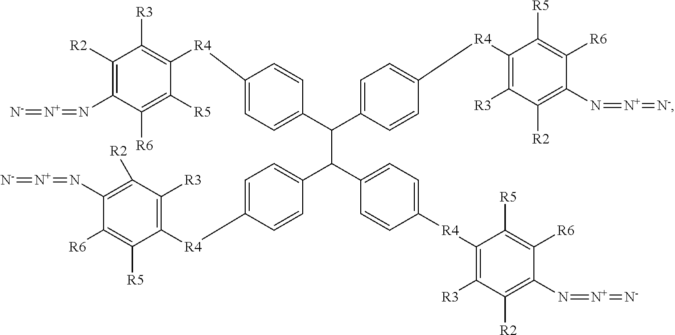

and the crosslinking agent includes four azide groups N3.

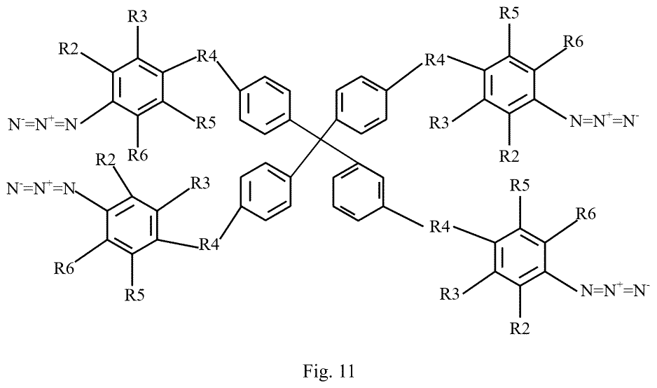

As shown in FIG. 11, when R is

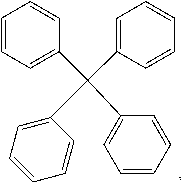 [SMILES: c1ccc(C(c2ccccc2)(c2ccccc2)c2ccccc2)cc1]

and the group R4 of R1 is connected with the reaction site of R, the chemical formula of the crosslinking agent can be expressed as

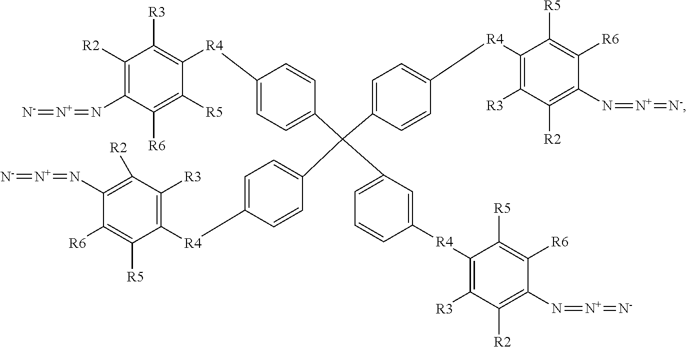

and the crosslinking agent includes four azide groups N3.

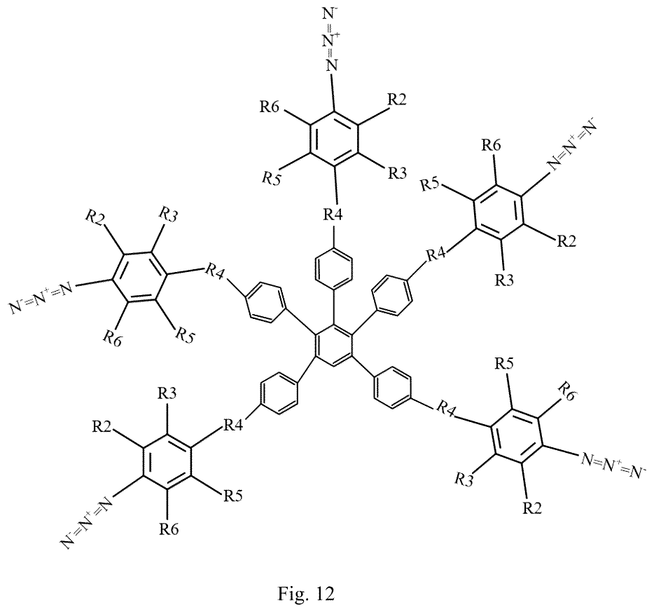

As shown in FIG. 12, when R is

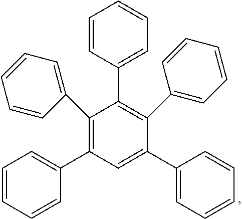 [SMILES: c1ccc(-c2cc(-c3ccccc3)c(-c3ccccc3)c(-c3ccccc3)c2-c2ccccc2)cc1]

and the group R4 of R1 is connected with the reaction site of R, the chemical formula of the crosslinking agent can be expressed as

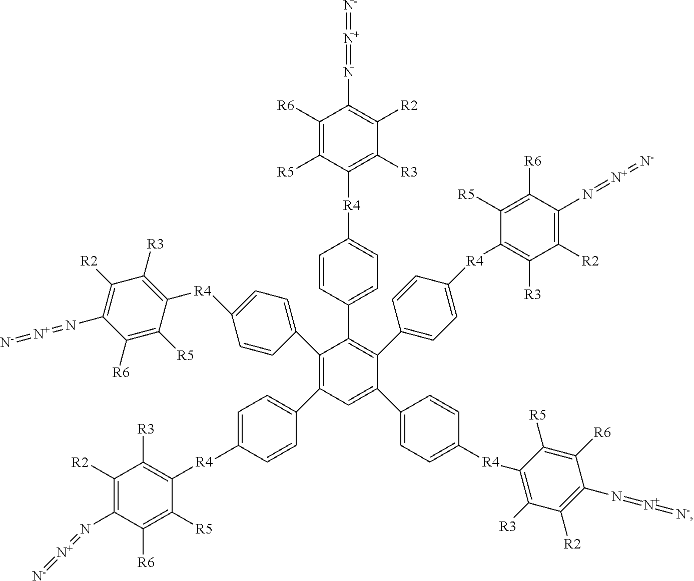

and the crosslinking agent includes five azide groups N3.

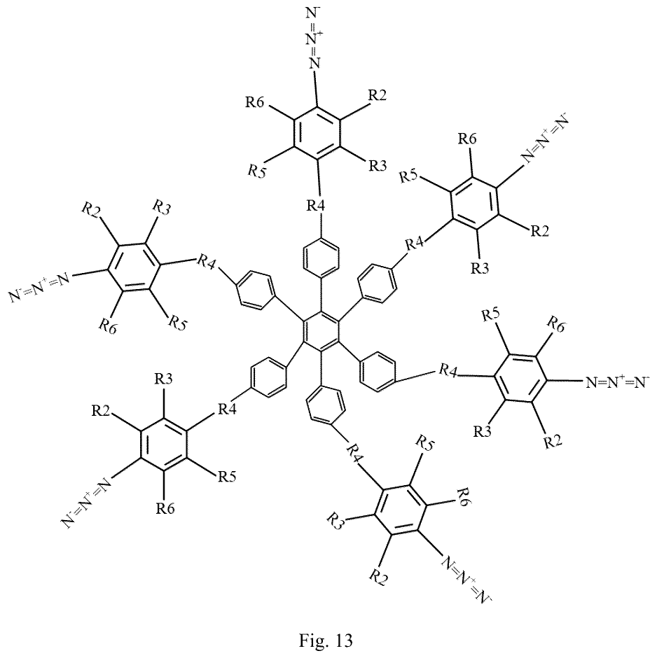

As shown in FIG. 13, when R is

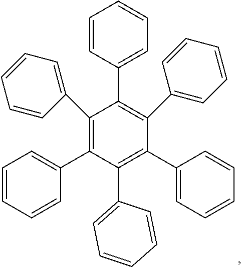 [SMILES: c1ccc(-c2c(-c3ccccc3)c(-c3ccccc3)c(-c3ccccc3)c(-c3ccccc3)c2-c2ccccc2)cc1]

and the group R4 of R1 is connected with the reaction site of R, the chemical formula of the crosslinking agent can be expressed as

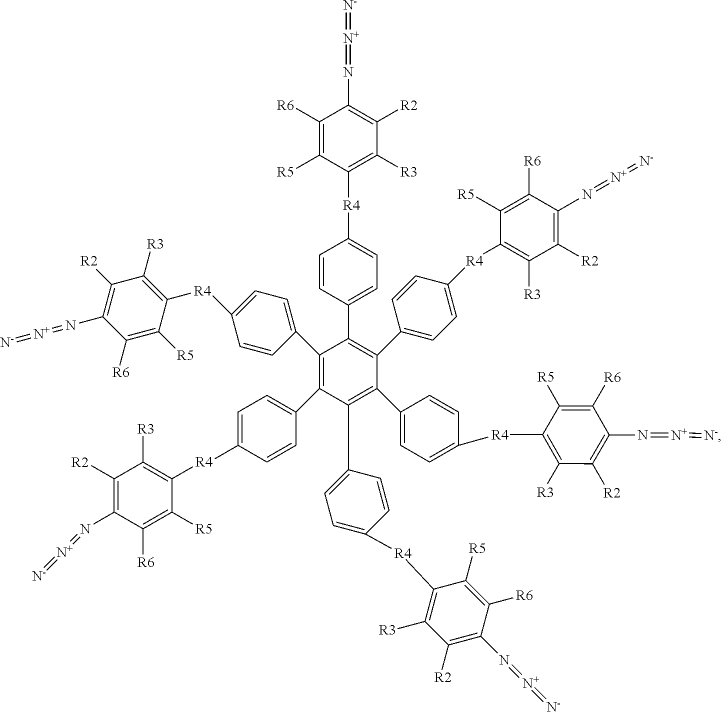

and the crosslinking agent includes six azide groups N₃.

In the chemical formula of FIGS. 7-13, as mentioned above, the group R4 may be a conjugated group or a non-conjugated group, and the remaining groups R2, R3, R5 and R6 can be the same or different from each other and can be fluorine atoms or hydrogen atoms respectively. Non-conjugated groups may include at least one of ester groups, amide groups, ether groups, imine groups and carbon-nitrogen double bonds, and conjugated groups may be, for example, carbon-carbon double bonds.

In some embodiments, when the group R is a polymer chain, the molecular weight of the general formula R—(R1)ₙ may be 10000~30000, for example, the molecular weight may be 10000, 20000, 30000, etc. Illustratively, the value of n may be any integer between 80 and 120, that is, a single polymer molecule may include 80 to 120 azide groups N₃, for example, it may include 80 azide groups N₃, 100 azide groups N₃, 120 azide groups N₃. The functional group of polymer chain includes at least one of carboxyl group, hydroxyl group and aldehyde group. In an example, the polymer chain may be polymethacrylic acid. In an alternative example, the polymer chain may be polyvinyl phenol.

In some embodiments, the chemical formula of R1 is

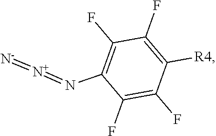

and R4 is connected with the reaction site on R. That is to say, in this chemical formula, the para group R4 of the azide group N₃ is connected with the reaction site on R, and the group R4 is a conjugated group or a non-conjugated group, and other groups R2, R3, R5 and R6 are all fluorine atoms. The hydrogen atoms in the ortho position and meta position of azide group N3 are replaced by fluorine atoms, which is beneficial to better stabilize nitrogen carbene radicals and promote the insertion reaction between crosslinking agent and carbon-hydrogen bonds of quantum dot ligands.

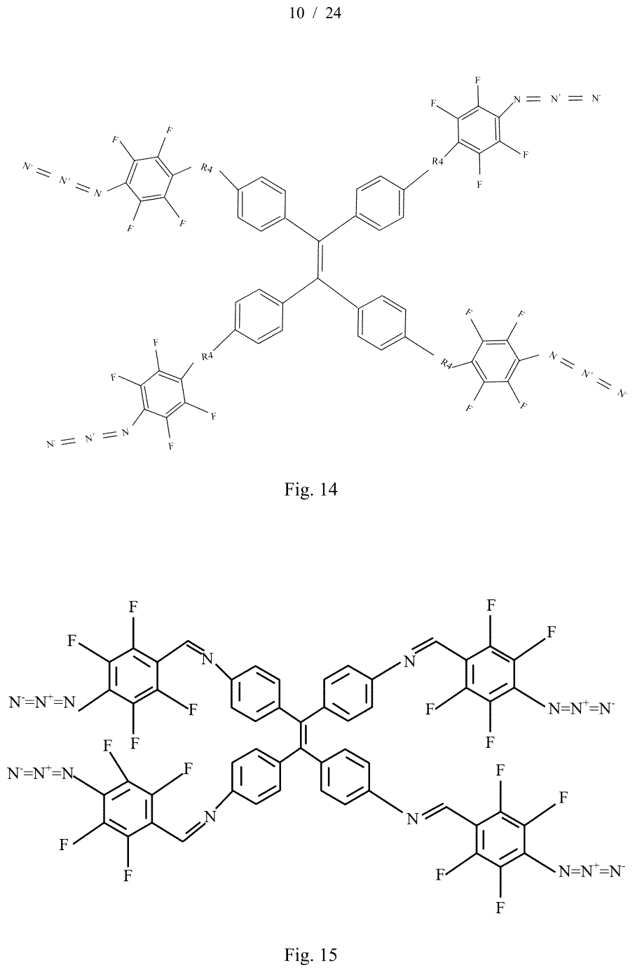

As shown in FIG. 14, in some embodiments, when R is

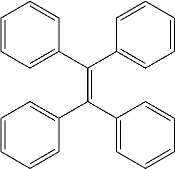 [SMILES: c1ccc(C(=C(c2ccccc2)c2ccccc2)c2ccccc2)cc1]

### and R1 is

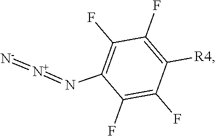

n in the general formula R—(R1)ₙ is equal to 4 and the chemical formula of the crosslinking agent is

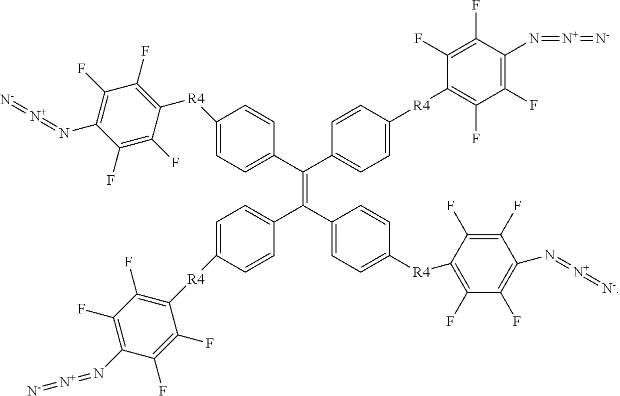

In this chemical formula, the middle mother nucleus R is selected as tetraethylene, which includes four reaction sites, so it can be connected with four groups R1, that is, the crosslinking agent includes four azide groups N₃. Since azide group N₃ has high photoinitiation efficiency, the crosslinking agent with tetrasubstituted azide group N₃ has good crosslinking degree, and the stability of quantum dot light emitting layer can be improved by using this crosslinking agent.

When the group R4 in the chemical formula of FIG. 14 is selected as a different group, the crosslinking agent may have a different molecular structure. Next, several crosslinking agents with different molecular structures and their preparation methods are listed.

As shown in FIG. 15, when R4 is a carbon-nitrogen double bond, the chemical formula of the crosslinking agent in FIG. 14 becomes

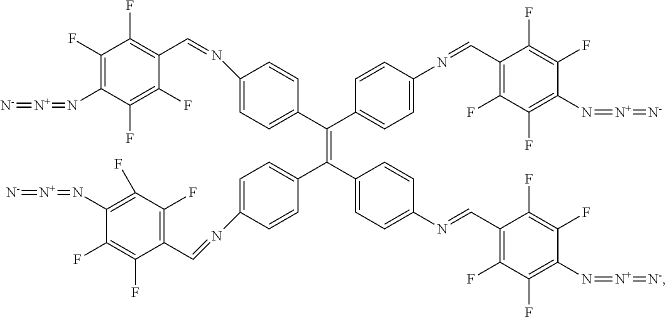

and the crosslinking agent may be abbreviated as TPE-4N₃.

FIG. 16 shows the synthesis route of TPE-4N₃. The synthesis method is roughly as follows: weighing a certain mass (for example, 392 mg) of the reactant

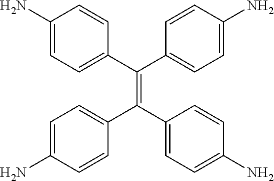 [SMILES: Nc1ccc(C(=C(c2ccc(N)cc2)c2ccc(N)cc2)c2ccc(N)cc2)cc1]

and a certain mass (for example, 860 mg) of the reactant

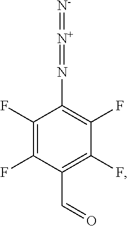 [SMILES: [N-]=[N+]=Nc1c(F)c(F)c(C=O)c(F)c1F]

placing the two reactants in a reaction container, adding methanol into the reaction container, and performing reflux reaction at 60° C. to obtain yellow solid powder. The yellow solid powder is washed with methanol and drained to obtain the product TPE-4N₃.

FIG. 17 shows the nuclear magnetic resonance hydrogen spectrum of TPE-4N₃, which shows that the molecular structure of the synthesized TPE-4N₃ is correct.

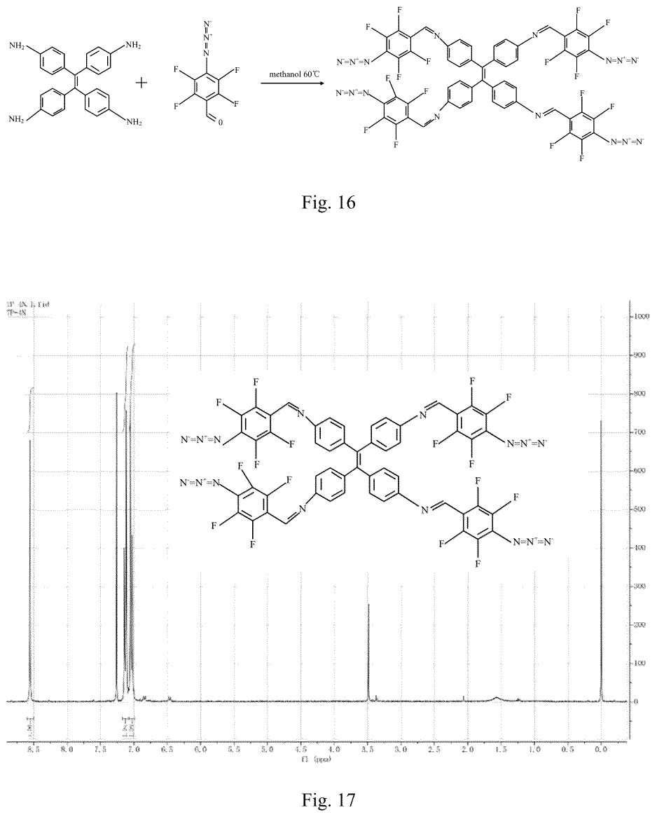

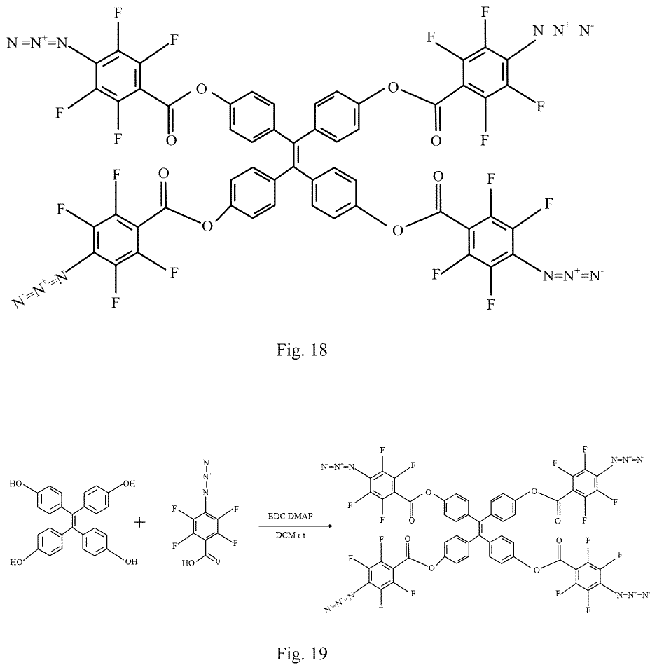

As shown in FIG. 18, when R4 is an ester group, the chemical formula of the crosslinking agent of FIG. 14 becomes

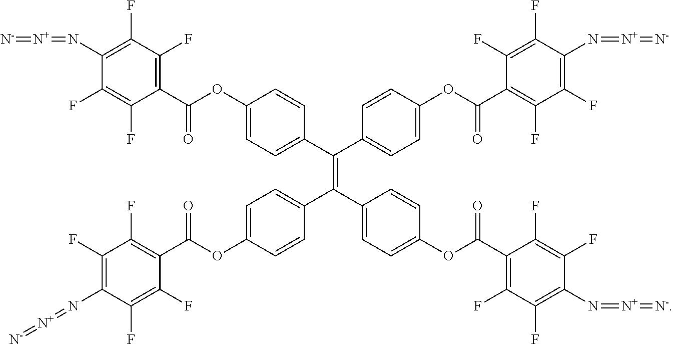

Although the azide group N3 shown in FIG. 18 is arranged in a para position with the ester group, as previously described, the azide group N3 may also be arranged in an ortho position or a meta position with the ester group.

FIG. 19 shows a synthesis route of the crosslinking agent of FIG. 18. The synthesis method is roughly as follows: weighing a certain mass of the reactant

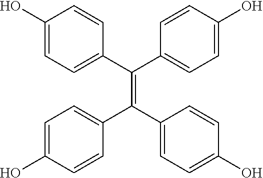 [SMILES: Oc1ccc(C(=C(c2ccc(O)cc2)c2ccc(O)cc2)c2ccc(O)cc2)cc1]

and a certain mass of the reactant

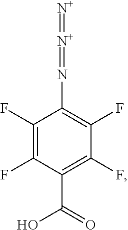 [SMILES: [NH2+]=[N+]=Nc1c(F)c(F)c(C(=O)O)c(F)c1F]

placing the two reactants in a reaction container, adding 1-ethyl-(3-dimethylaminopropyl) carbodiimide (EDC for short), 4-dimethylaminopyridine (DMAP for short) and dichloromethane (DCM for short) into the reaction container, and reacting at room temperature for a period of time to finally obtain the product

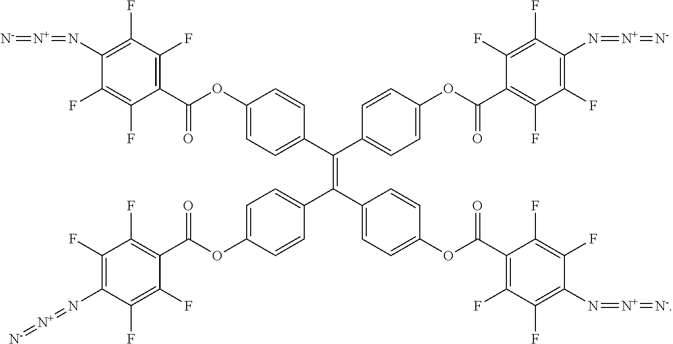

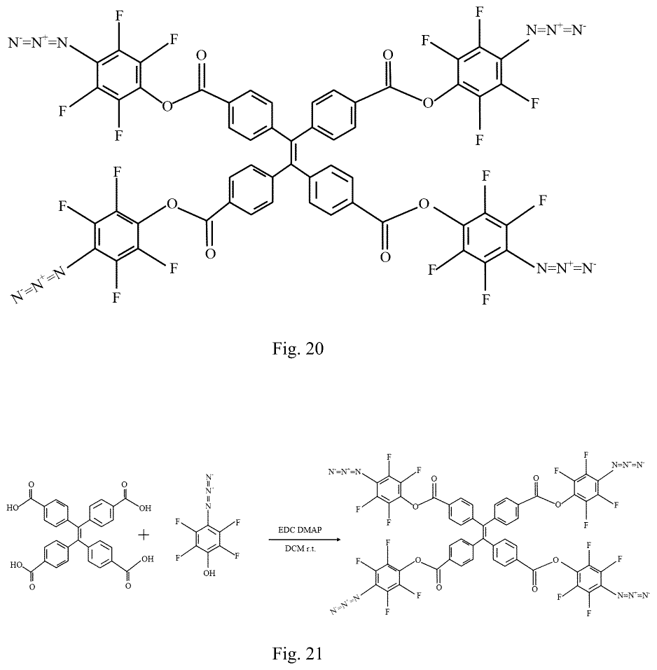

As shown in FIG. 20, when R4 is an ester group, the chemical formula of the crosslinking agent in FIG. 14 may also be

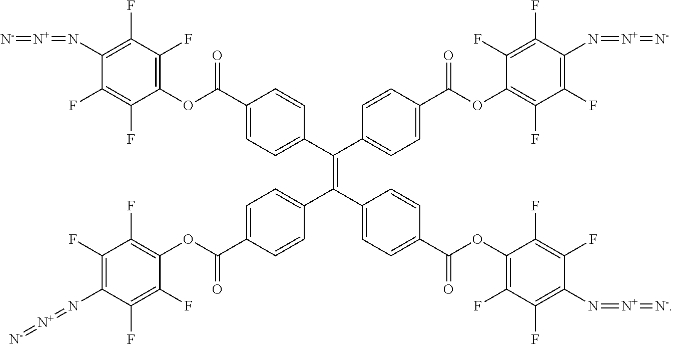

FIG. 21 shows a synthesis route of the crosslinking agent of FIG. 20. The synthesis method is roughly as follows: weighing a certain mass of the reactant

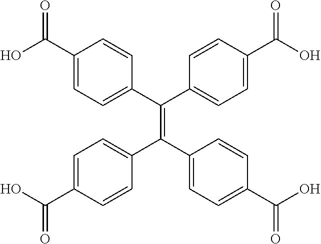 [SMILES: O=C(O)c1ccc(C(=C(c2ccc(C(=O)O)cc2)c2ccc(C(=O)O)cc2)c2ccc(C(=O)O)cc2)cc1]

and a certain mass of the reactant

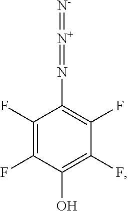 [SMILES: [N-]=[N+]=Nc1c(F)c(F)c(O)c(F)c1F]

placing the two reactants in a reaction container, adding EDC, DMAP and DCM into the reaction container, and reacting at room temperature for a period of time to finally obtain the product

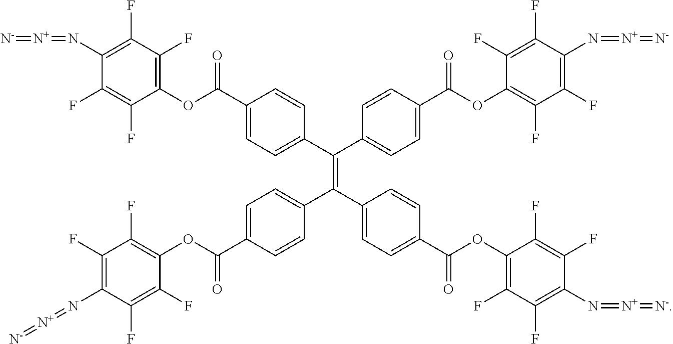

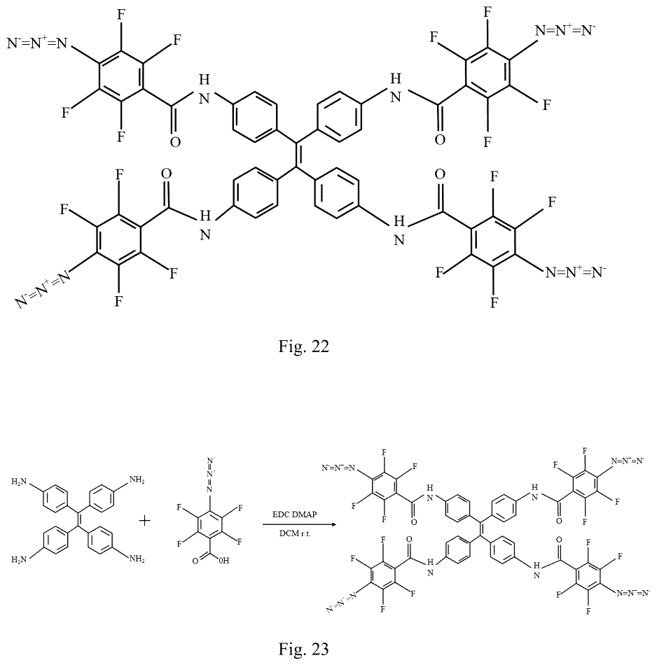

As shown in FIG. 22, when R4 is an amide group, the chemical formula of the crosslinking agent of FIG. 14 becomes

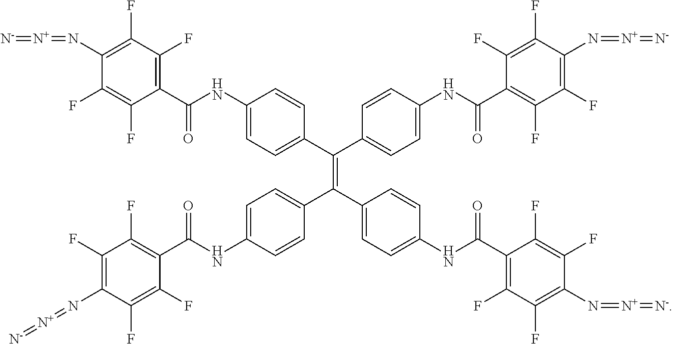

FIG. 23 shows a synthesis route of the crosslinking agent of FIG. 22. The synthesis method is roughly as follows: weighing a certain mass of the reactant

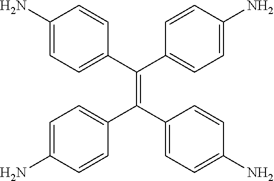 [SMILES: Nc1ccc(C(=C(c2ccc(N)cc2)c2ccc(N)cc2)c2ccc(N)cc2)cc1]

and a certain mass of the reactant

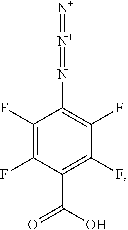 [SMILES: [NH2+]=[N+]=Nc1c(F)c(F)c(C(=O)O)c(F)c1F]

placing the two reactants in a reaction container, adding EDC, DMAP and DCM into the reaction container, and reacting at room temperature for a period of time to finally obtain the product

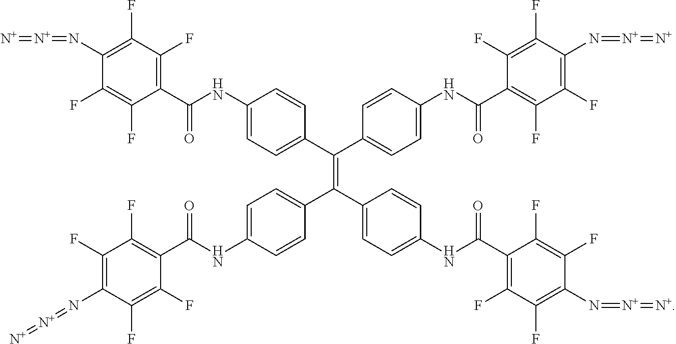

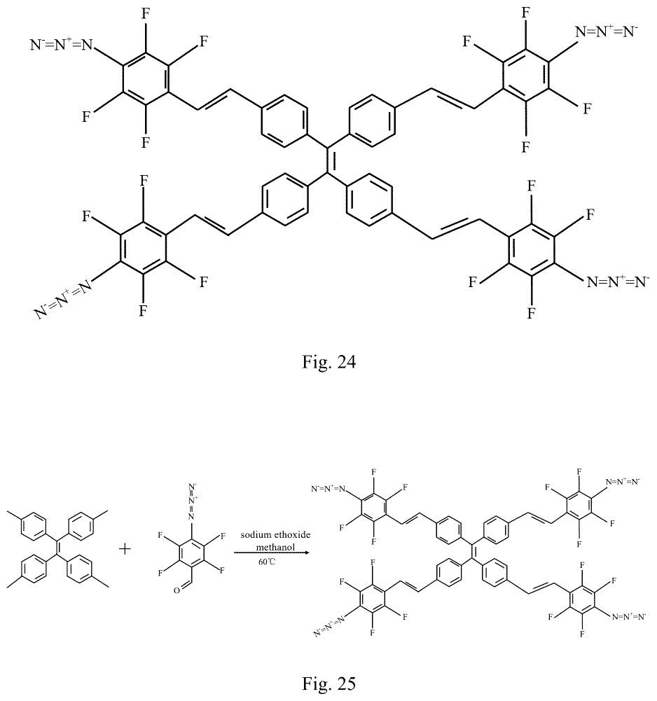

As shown in FIG. 24, when R4 is a carbon-carbon double bond, the chemical formula of the crosslinking agent in FIG. 14 becomes

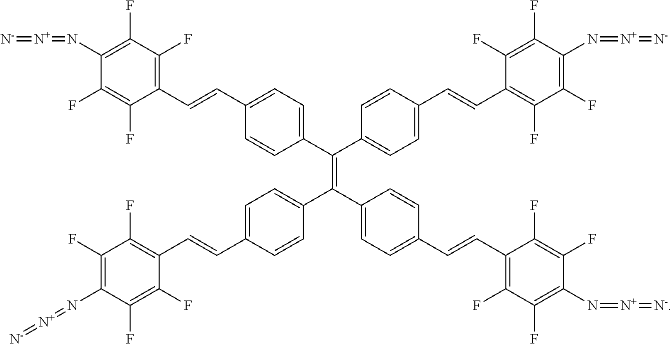

FIG. 25 shows a synthesis route of the crosslinking agent of FIG. 24. The synthesis method is roughly as follows: weighing a certain mass of the reactant

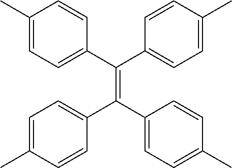

and a certain mass of the reactant

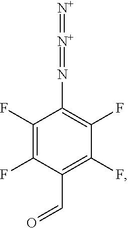 [SMILES: [NH2+]=[N+]=Nc1c(F)c(F)c(C=O)c(F)c1F]

placing the two reactants in a reaction container, adding sodium ethoxide and methanol into the reaction container, and performing reflux reaction at 60° C. for a period of time to finally obtain the product

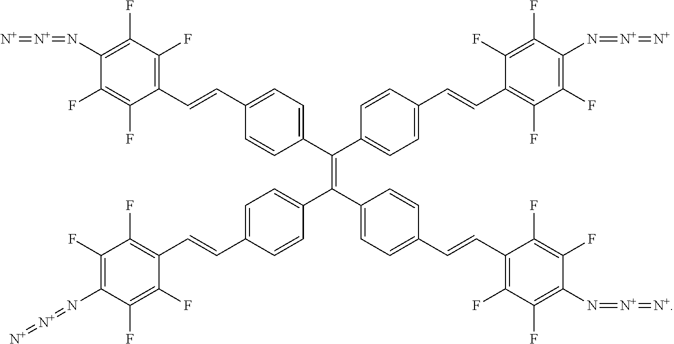

The selection of the group R4 of the crosslinking agent mainly considers the solubility, the source of raw materials and the difficulty of post-treatment. In terms of solubility, according to the principle of similar miscibility between molecules, the crosslinking agent is guaranteed to have good compatibility with quantum dots. For example, when the solvent of quantum dots contains ester groups, the group R4 of the crosslinking agent can be selected as the ester group. When the solvent of quantum dots contains amide groups, the group R4 of the crosslinking agent can be selected as amide group. When the solvent of quantum dots contains carbon-nitrogen double bonds, the group R4 of the crosslinking agent can be selected as carbon-nitrogen double bonds. When the solvent of quantum dots contains carbon-carbon double bonds, the group R4 of the crosslinking agent can be selected as carbon-carbon double bonds. When the solvent of quantum dots contains ether group, the group R4 of the crosslinking agent can be selected as ether group.

In addition, it should be noted that the molecular structure of the crosslinking agent can be symmetric or asymmetric, which is not limited by the embodiments of the present disclosure.

In the crosslinking agents provided by various embodiments of the present disclosure, the group R has at least three reaction sites that can be connected with the group R1, so each group R can be connected with at least three groups R1, and each R1 includes at least one azide group N₃, so the crosslinking agent includes at least three azide groups N3. Because the photoinitiation efficiency of the azide group N₃ is relatively high, the crosslinking agent with multi-substituted azide group N₃ provided by various embodiments of the present disclosure has a high degree of crosslinking. When the crosslinking agent reacts with quantum dots, the formed quantum dot light emitting layer has high stability, which is helpful to enhance the stability of the quantum dot light emitting layer in the exposed area. Under the action of the developing solution, the part of the quantum dot light emitting layer in the exposure area will not be damaged by the developing solution, and the expected pattern shape can be well formed. At the same time, the part of the quantum dot light emitting layer in the unexposed area can be washed away by the developing solution without residue, thus avoiding the color mixing phenomenon caused by the residue of quantum dots. By using the crosslinking agents provided by the embodiments of the present disclosure, the problem of quantum dot residue can be solved, and high-quality quantum dot patterns can be obtained.

FIG. 26 shows a schematic structural diagram of a quantum dot according to an embodiment of the present disclosure. As shown in FIG. 26, the quantum dot comprises a quantum dot body and a ligand modified on the quantum dot body through coordination, and the ligand comprises carbon-hydrogen bonds and can crosslink with the crosslinking agent described in any of the previous embodiments under light radiation. Specifically, because the crosslinking agent includes azide group N₃, which can generate nitrogen carbene radical under light radiation, its activity is relatively high. Therefore, under light radiation, the crosslinking agent can undergo the nitrogen-hydrogen insertion reaction with carbon-hydrogen bonds of ligands on the surface of quantum dots to form a crosslinked structure.

The ligand on the surface of the quantum dot can be any suitable ligand. In some embodiments, the ligand can be selected from any one of oleylamine, isooctyl mercaptan, mercaptoacetic acid, mercaptopropionic acid, oleic acid and 2-mercaptoethyl Boc amine. The ligand on the surface of the quantum dot can fill the defects on the surface of the quantum dot, thus improving the stability and quantum yield of the quantum dot.

In some embodiments, the quantum dots are configured to emit red light or blue light (or called red quantum dots or blue quantum dots), and the ligands on the surface of the quantum dots are oleic acid, and the molecular structure of oleic acid is

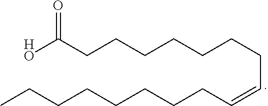 [SMILES: CCCCCCCC/C=C\CCCCCCCC(=O)O]

After the red quantum dots or blue quantum dots with oleic acid ligands crosslink with the above described crosslinking agents, a high-quality quantum dot patterned layer can be obtained.

In some alternative embodiments, the quantum dots are configured to emit green light (or called green quantum dots), and the ligands on the surface of the quantum dots is 2-mercaptoethyl Boc amine, and the chemical formula of 2-mercaptoethyl Boc amine is

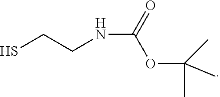

After the green quantum dots with 2-mercaptoethyl Boc amine ligands crosslink with the above described crosslinking agents, a high quality quantum dot patterned layer can be obtained.

The quantum dots provided by various embodiments of the present disclosure may be any suitable quantum dots, comprising but not limited to any of IIB-VIA quantum dots, IIIA-VA quantum dots, IV A-VIA quantum dots, core-shell quantum dots and ABX₃ perovskite quantum dots. In ABX₃ perovskite quantum dots, A is one or more of CH₃NH₃⁺(methylamine), NH₂CH═NH₂ (formamidine) and Cs⁺, B is one or two of Pb²⁺ and Sn²⁺, X is one or more of Cl⁻, Br⁻ and I⁻, and ABX₃ perovskite quantum dots comprise CH₃NH₃PbBr₃, CH₃NH₃PbCl₃, CH₃NH₃PbI₃, CsPbBr₃, CsPbCl₃ and CsPbI₃.

Exemplary, the IIB-VIA quantum dots are selected from one or more binary compounds such as CdSe, CdTe, ZnS, ZnSe, ZnTe, ZnO, HgS, HgSe, HgTe, MgSe and MgS; ternary compounds such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS or mixtures thereof; and quaternary compounds such as HgZnTeS, CdZnSeS, CdZnSeTe, CdZnSTe, CdHgSeS, CdHgSeTe, CdHgSTe, HgZnSeS, HgZnSeTe, HgZnSTe, or mixtures thereof, but not limited thereto.

Group IIIA-VA quantum dots are selected from binary compounds such as GaN, GaP, GaAs, GaSb, AlN, AlP, AlAs, AlSb, InN, InP, InAs, InSb, or mixtures thereof; ternary compounds such as GaNP, GaNAs, GaNSb, GaPAs, GaPSb, AlNP, AlNAs, AlNSb, AlPAs, AlPSb, InNP, InNAs, InNSb, InPAs, InPSb, or mixtures thereof; and quaternary compounds such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPASb, or mixtures thereof, but not limited thereto.

IVA-VIA quantum dots are selected from binary compounds such as SnS, SnSe, SnTe, PbS, PbSe, PbTe, or mixtures thereof; ternary compounds such as SnSeS, SnSeTe, SnSTe, PbSeS, PbSeTe, PbSTe, SnPbSe, SnPbSe, SnPbTe, or mixtures thereof; and quaternary compounds such as SnPbSSe, SnPbSeTe, SnPbSTe, or mixtures thereof, but not limited thereto. IVA-VIA quantum dots are, for example, selected from elementary substance (unitary) semiconductors such as Si, Ge, or mixtures thereof; and binary semiconductor compounds such as SiC, SiGe, and mixtures thereof, but not limited thereto.

Quantum dots with core-shell structure mean that one material is the core material and the other is the shell material. For example, the quantum dot is CdS/ZnS, which means that the core material of the quantum dot is CdS and the shell material is ZnS.

In other embodiments, quantum dots may be other nano-scale materials, such as nanorods, nanosheets. Components of other nano-scale materials may comprise at least one of CdS, CdSe, CdTe, ZnSe, InP, PbS, CuInS₂, ZnO, CsPbCl₃, CsPbBr₃, CsPhI₃, CdS/ZnS, CdSe/ZnS, ZnSe, InP/ZnS, PbS/ZnS, InAs, InGaAs, InGaN, GaN, ZnTe, Si, Ge and C.

For example, the quantum dots may comprise cadmium free quantum dots. Cadmium-free quantum dots are quantum dots that do not comprise cadmium (Cd). Cadmium (Cd) can cause serious environmental/health problems, so non-Cd-based quantum dots can be effectively used.

According to yet another aspect of the present disclosure, a quantum dot composition is provided. The quantum dot composition comprises a mixed solution comprising the quantum dots with ligands described above, a crosslinking agent described in any of the previous embodiments, and a solvent. The concentration of quantum dots in the mixed solution may be about 20~35 mg/mL, such as 20 mg/mL, 30 mg/mL, 35 mg/mL. The concentration of crosslinking agent in the mixed solution may be about 0.1~2.0 mg/mL, such as 0.1 mg/mL, 0.5 mg/mL, 1.0 mg/mL, 1.5 mg/mL, 2.0 mg/mL.

In the quantum dot composition, as mentioned above, the quantum dot comprises a quantum dot body and a ligand modified on the quantum dot body through coordination, and the ligand comprises carbon-hydrogen bonds. The ligand may be selected from, for example, oleylamine, isooctyl mercaptan, mercaptoacetic acid, mercaptopropionic acid, oleic acid and 2-mercaptoethyl Boc amine. After the mixed solution is cured, under light irradiation, the azide group N₃ of the crosslinking agent may undergo the nitrogen-hydrogen insertion reaction with the carbon-hydrogen bond of the ligand on the surface of the quantum dot to form a crosslinked structure.

The solvent in the quantum dot composition may be any suitable type of solvent. In some embodiments, the solvent may be, for example, any one of propylene glycol methyl ether acetate (PGMEA), toluene, chlorobenzene, octane, water and alcohol solvents. Quantum dots and the crosslinking agent have good solubility in PGMEA, and the solvent is pollution-free to the environment.

According to yet another aspect of the present disclosure, a quantum dot light emitting layer is provided. The quantum dot light emitting layer comprises a plurality of quantum dots, at least some of the plurality of quantum dots comprise quantum dot bodies and ligands modified on the quantum dot bodies through coordination, and the quantum dot light emitting layer is generated by crosslinking quantum dots with ligands and a crosslinking agent described in any of the previous embodiments.

In some embodiments, when the surface of each quantum dot has a ligand, the general formula of the quantum dot light emitting layer can be expressed as R—(R1′-R7-QD)ₙ, where QD represents a quantum dot, R7 represents a ligand on the surface of the quantum dot and comprises carbon-hydrogen bonds, R is the mother core structure of a crosslinking agent, R comprises at least three reaction sites that can be connected with R1, and R may be

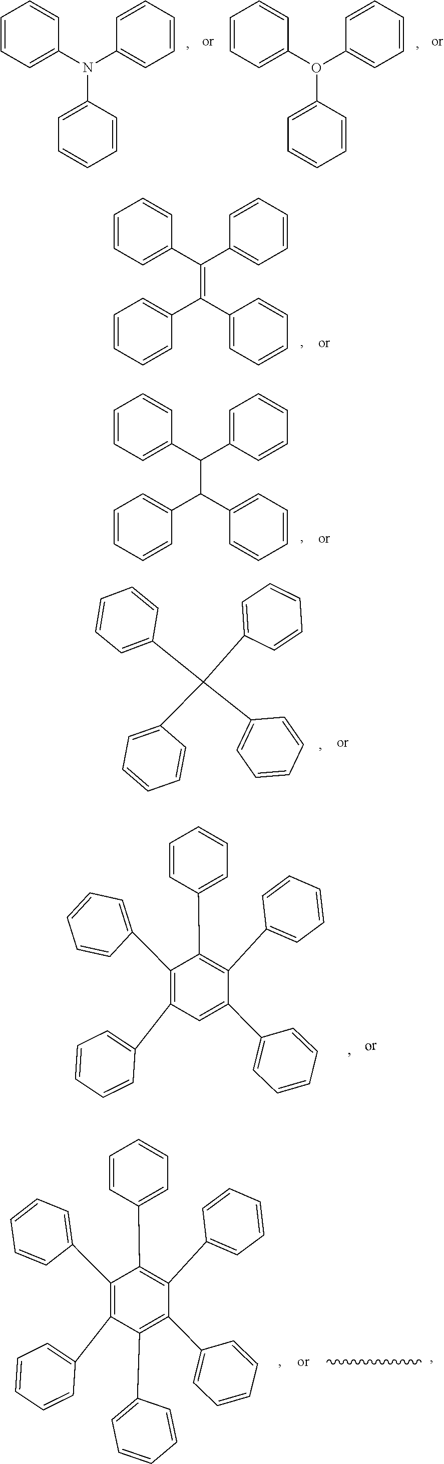

or other suitable groups.

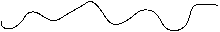

represents a polymer chain, and the functional group of the polymer chain comprises at least one of carboxyl group, hydroxyl group and aldehyde group. n is a positive integer greater than or equal to 3, and the value of n is equal to the number of reaction sites on R. R1′ represents a group after the azide group N₃ on R1 is changed into NH through the nitrogen-hydrogen insertion reaction. In some embodiments, the chemical formula of R1′ can be expressed as

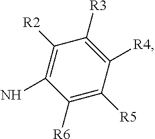

wherein any one of R2~R6 is a conjugated group or a non-conjugated group, and the conjugated group or the non-conjugated group is connected with the reaction site on R, and the remaining groups in R2~R6 except the group connected with the reaction site on R can be the same or different from each other, and can be respectively selected from fluorine atoms or hydrogen atoms. The non-conjugated group may include at least one of ester group, amide group, ether group, imine group and carbon-nitrogen double bond. The conjugated group may include a carbon-carbon double bond.

The quantum dot light emitting layer may be applied to the field of electroluminescence and may also be applied to the field of photoluminescence.

In the field of electroluminescence, for example, the quantum dot light emitting layer may be used as the light emitting layer in a quantum dot light emitting diode device. Under the action of an electric field, electrons and holes generated in the device are transmitted to the quantum dot light emitting layer, and are recombined into excitons in the quantum dot light emitting layer to generate energy level transitions, thereby emitting light.

In the field of photoluminescence, quantum dot light emitting layer may be integrated in backlight. Alternatively, the quantum dot light emitting layer may be used as a color film and integrated inside the panel, and may be applied to a liquid crystal display panel, an organic light emitting diode display panel, or a submillimeter organic light emitting diode display panel. Alternatively, the quantum dot light emitting layer may be arranged on the light exit side of the organic light emitting diode display panel emitting blue light, and emit three colors of light, red, green and blue, under the excitation of blue light, thereby realizing full-color display.

FIG. 27 shows a chemical formula of a quantum dot light emitting layer. When R is

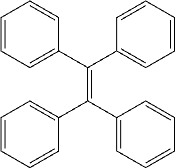 [SMILES: c1ccc(C(=C(c2ccccc2)c2ccccc2)c2ccccc2)cc1]

### and R1′ is

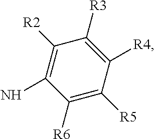

and R4 is connected with the reaction site on R, the general formula R—(R1′-R7-QD)ₙ may be expressed as

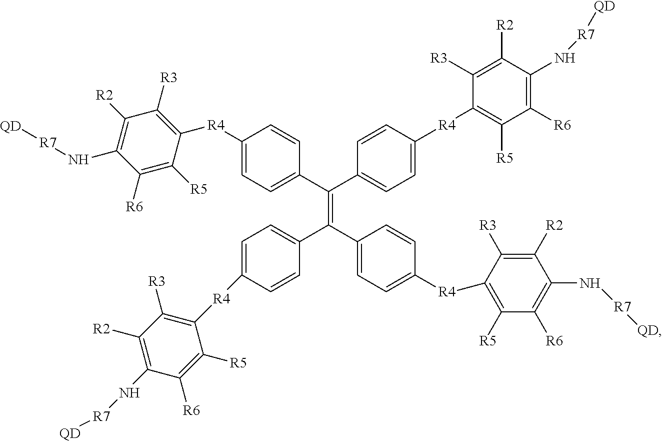

where QD represents a quantum dot, R7 is a ligand on the surface of the quantum dot and comprises carbon-hydrogen bonds, R4 is a conjugated group or a non-conjugated group, and R2, R3, R5 and R6 may be the same or different from each other and may be selected from fluorine atoms or hydrogen atoms respectively. Conjugated groups may include, for example, carbon-carbon double bonds, and unconjugated groups may include, for example, at least one of ester groups, amide groups, ether groups, imine groups, and carbon-nitrogen double bonds.

It can be seen from the chemical formula of the light emitting layer of the quantum dot that the mother nucleus R of the crosslinking agent used for crosslinking reaction with the quantum dot is selected as tetraethylene, which can be connected with four groups R1 since it comprises four reaction sites, that is, the crosslinking agent comprises four azide groups N₃. When the crosslinking agent reacts with the quantum dot under light radiation, the azide group N₃ of the crosslinking agent reacts with the carbon-hydrogen bond of the ligand R7 on the surface of the quantum dot, and the azide group N₃ on the group R1 becomes NH, the NH connected with the ligand R7, thus forming the quantum dot light emitting layer with crosslinking structure. The crosslinking agent with tetrasubstituted azide group N₃ can improve the crosslinking degree between the crosslinking agent and quantum dots, and the formed quantum dot light emitting layer has high stability. When developing such a quantum dot light emitting layer with a developing solution, the part of the quantum dot light emitting layer in the exposure area will not be damaged by the developing solution, and the expected pattern shape can be well formed. At the same time, the part of the quantum dot light emitting layer in the unexposed area can be washed away by the developing solution without residue, thus avoiding the color mixing phenomenon caused by quantum dot residue. In this way, the problem of quantum dot residue can be solved and high quality quantum dot patterns can be obtained.

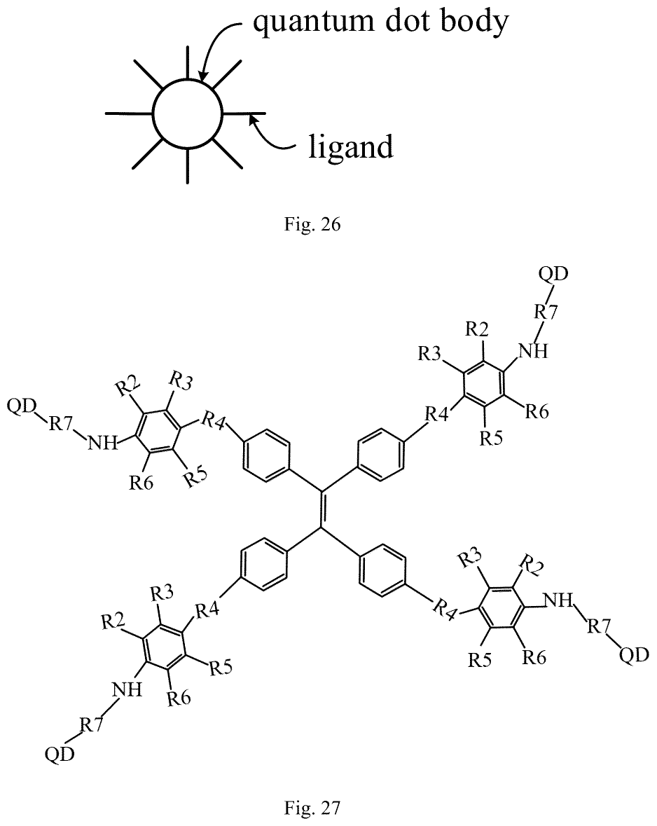

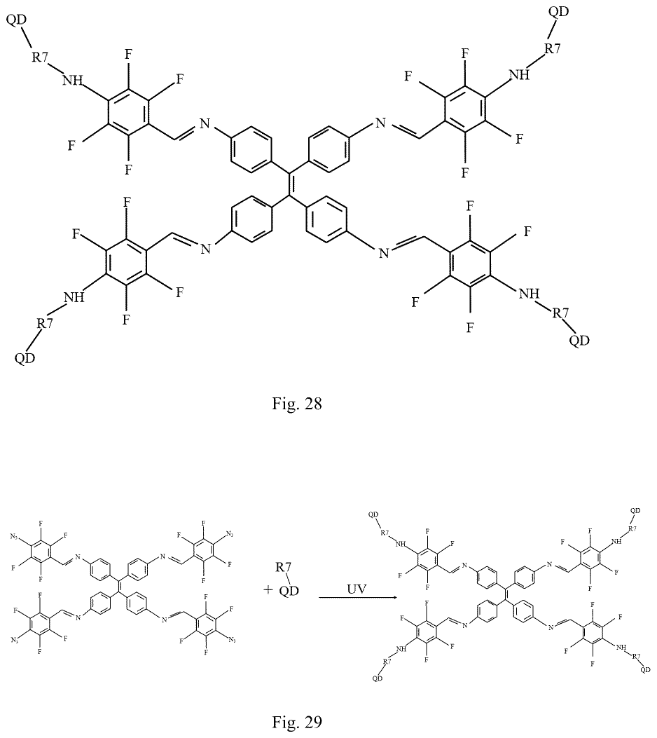

FIG. 28 shows a chemical formula of a quantum dot light emitting layer, the chemical formula is

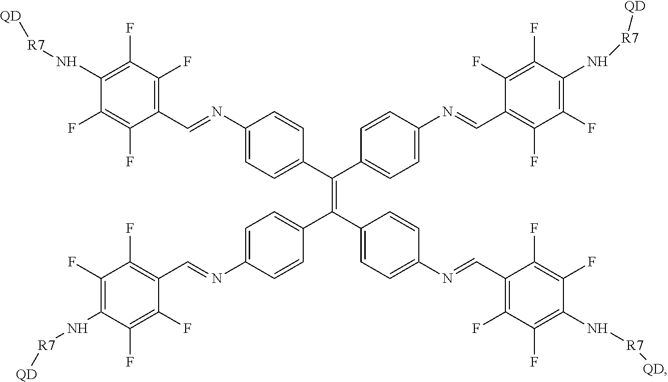

where QD represents a quantum dot and R7 represents a ligand on the surface of the quantum dot. R7 is oleic acid, and its chemical formula is

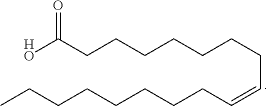 [SMILES: CCCCCCCC/C=C\CCCCCCCC(=O)O]

The quantum dot light emitting layer may be a red quantum dot light emitting layer or a blue quantum dot light emitting layer.

FIG. 29 shows a synthesis route of the quantum dot light emitting layer of FIG. 28, and the preparation process of the quantum dot light emitting layer of FIG. 28 will be described below with reference to FIG. 29.

Firstly, a solution of the electron transport layer was spin-coated on the substrate to form the electron transport layer. The electron transport layer may be formed of any suitable material, for example, the material of the electron transport layer may be nano-particle type zinc oxide (ZnO), or metal-doped nano-zinc oxide, or Sol-Gel type zinc oxide (ZnO). For example, metal-doped nano-zinc oxide may include zinc oxide (ZnO) doped with magnesium (Mg), aluminum (Al), zirconium (Zr) or yttrium (Y). In some embodiments, the material of the electron transport layer is ZnO with nanoparticle structure, for example, ZnO with nanoparticle structure may be formed by sol-gel method. In some embodiments, the concentration of ZnO in the solution may be 75 mg/mL, the speed of spin coating the solution on the substrate may be 3000 rpm, and the heating temperature required in the sol-gel process may be about 300° C.

Then, a mixed solution including a crosslinking agent, quantum dots with ligands, and a solvent was prepared. The crosslinking agent is the aforementioned crosslinking agent

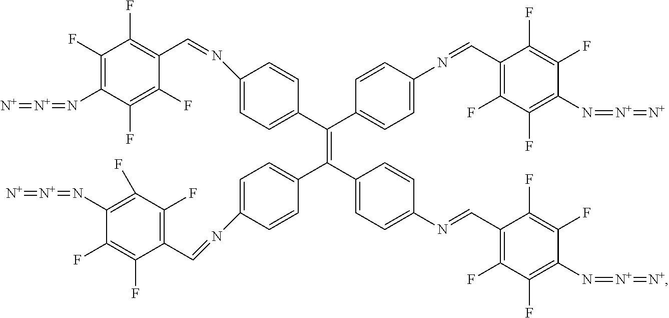

abbreviated as TPE-4N₃, and the ligand R7 on the surface of quantum dot QD is oleic acid, and the quantum dot with ligand can be abbreviated as QD-R7. Since quantum dots QD-R7 with oleic acid ligand have good solubility in chlorobenzene (for example, solubility>30 mg/mL) and crosslinking agent TPE-4N₃ also has good solubility in chlorobenzene (for example, solubility>5 mg/mL), chlorobenzene can be selected as the solvent to make the crosslinking agent TPE-4N₃ and quantum dots QD-R7 better compatible. In some embodiments, the concentration of quantum dots QD-R7 in the mixed solution may be designed as 30 mg/mL, and the concentration of crosslinking agent TPE-4N₃ in the mixed solution may be designed as 1 mg/mL. The mixed solution was spin-coated on the electron transport layer and cured to form a cured layer. Then, ultraviolet light was used for exposure, and the exposure time may be about 60 s, for example. The four azide groups N₃ of the crosslinking agent TPE-4N₃ can generate nitrogen carbene free radicals under ultraviolet irradiation, which has high activity and can undergo the nitrogen-hydrogen insertion reaction with the carbon-hydrogen bond of oleic acid ligand R7 on the surface of quantum dots. After the insertion reaction of nitrogen and hydrogen, the azide group N₃ becomes NH, which is connected with the ligand R7, thus forming the quantum dot light emitting layer with crosslinking structure.

It should be noted that oleic acid includes multiple carbon-hydrogen bonds, and the azide group N₃ of the crosslinking agent TPE-4N₃ can undergo the nitrogen-hydrogen insertion reaction at any carbon-hydrogen bond position of oleic acid, and the insertion reaction position is not limited. Therefore, in the chemical formula

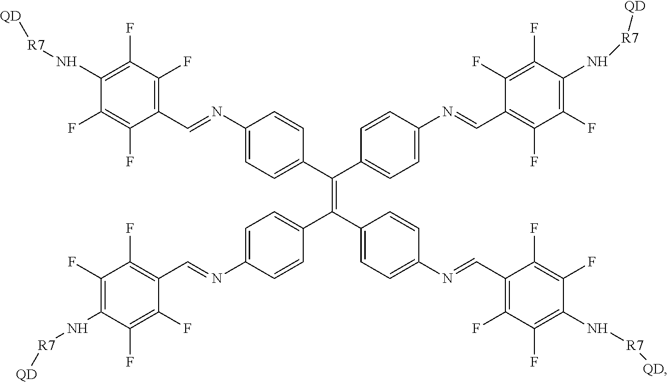

NH can be connected with any carbon atom of oleic acid ligand R7.

In an example, the quantum dot light emitting layer is a red quantum dot light emitting layer for emitting red light.

Then, the exposed red quantum dot light emitting layer was developed by developing solution to obtain a patterned red quantum dot light emitting layer.

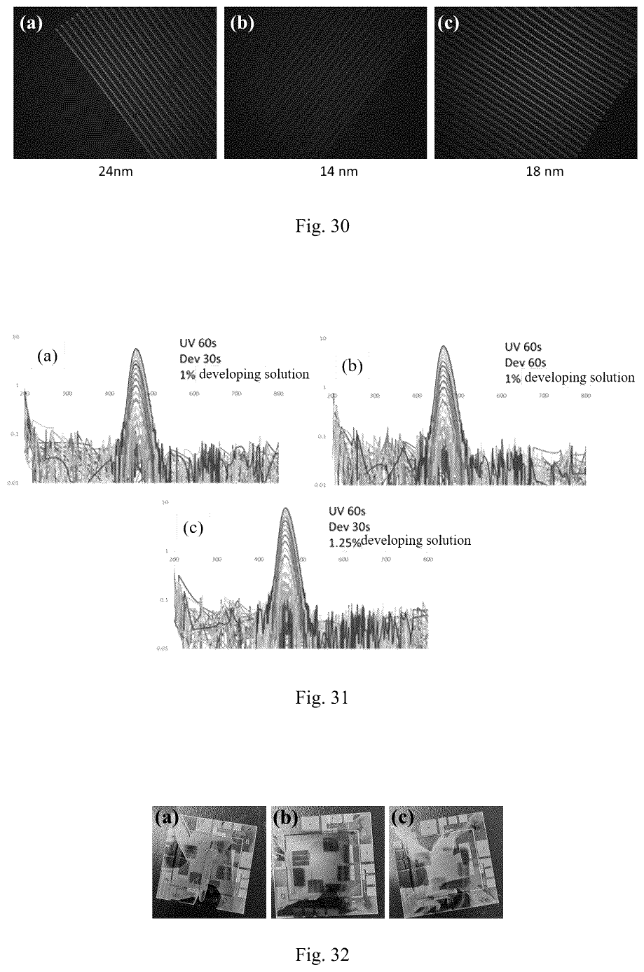

FIG. 30 explores the effects of different concentrations of developing solution and different development time on quantum dot patterns. FIG. 30(a) shows a photo of the light-induced patterning of the red quantum dot light emitting layer, in which the oblique straight line indicates the shape of the patterned red quantum dot light emitting layer in the exposed area in the lit state, and the black area indicates the unexposed area. In FIG. 30(a), the thickness of the red quantum dot light emitting layer is 24 nm, the corresponding developing solution concentration is 1%, and the development time is 30 s. FIG. 30(b) shows a photo of the light-induced patterning of the red quantum dot light emitting layer, in which the oblique straight line indicates the shape of the patterned red quantum dot light emitting layer in the exposed area in the lit state, and the black area indicates the unexposed area. In FIG. 30(b), the thickness of the red quantum dot light emitting layer is 14 nm, the corresponding developing solution concentration is 1%, and the development time is 60 s. FIG. 30(c) shows a photo of the light-induced patterning of the red quantum dot light emitting layer, in which the oblique straight line indicates the shape of the patterned red quantum dot light emitting layer in the exposed area in the lit state, and the black area indicates the unexposed area. In FIG. 30(c), the thickness of the red quantum dot light emitting layer is 18 nm, the corresponding developing solution concentration is 1.25%, and the development time is 30 s.

As can be seen from FIGS. 30(a), 30(b) and 30(c), under three different development conditions, the part of the red quantum dot light emitting layer in the exposure area has a perfect pattern shape and is not damaged by the developing solution. At the same time, the parts of the red quantum dot light emitting layer in unexposed areas (such as green and blue sub-pixel regions) are all washed away by the developing solution, and no red quantum dots remain. This shows that the red quantum dot light emitting layer has high stability, and under the action of developing solution, it can not only form high-quality quantum dot patterns in exposed areas, but also avoid quantum dot residues in unexposed areas and avoid color mixing.

The reason why the red quantum dot light emitting layer has high stability is that the red quantum dot light emitting layer is generated by crosslinking the crosslinking agent TPE-4N₃ and red quantum dots containing oleic acid ligands, and each crosslinking agent molecule TPE-4N₃ has four azide groups N₃. Since the azide groups N₃ have high photoinitiation efficiency and low exposure dose, and can generate nitrogen carbene free radicals under light radiation, the crosslinking agent TPE-4N₃ has high activity. When the crosslinking agent TPE-4N₃ and the red quantum dots containing oleic acid ligands crosslink under light radiation, the crosslinking agent TPE-4N₃ can undergo the nitrogen-hydrogen insertion reaction with the carbon-hydrogen bonds of the oleic acid ligands on the surface of the quantum dots to form a stable crosslinking structure, so that the formed red quantum dot light emitting layer has high stability and will not be destroyed by the developing solution, thus a high-quality red quantum dot pattern can be formed in the exposed area. Moreover, it further shows that the crosslinking agent TPE-4N₃ is an ideal crosslinking agent.

In order to further verify that there is no red quantum dot residue in the unexposed area, green quantum dot patterns and blue quantum dot patterns are further prepared on the basis of the above three red quantum dot patterns, and then the hole transport layer material and the electrode material were evaporated, and the devices were packaged to form three kinds of quantum dot light emitting devices.

FIG. 31 shows the electrospectrograms of green sub-pixels of three kinds of quantum dot light emitting devices, in which FIG. 31(a) corresponds to the experimental conditions of FIG. 30(a), that is, ultraviolet exposure for 60 s, developing solution concentration of 1% and development time of 30s; FIG. 31(b) corresponds to the experimental conditions of FIG. 30(b), that is, ultraviolet exposure for 60 s, developing solution concentration of 1% and development time of 60s; FIG. 31(c) corresponds to the experimental conditions of FIG. 30(c), that is, ultraviolet exposure for 60 s, developing solution concentration of 1.25% and development time of 30s. The green sub-pixels of quantum dot light emitting device are tested by electro-testing. With the gradual increase of voltage (for example, from 4V to 8V), it is found that the three kinds of electrospectrograms only have a peak at the position corresponding to the green wavelength, but there is no peak at the position corresponding to the red wavelength. This shows that there are only green quantum dots in the green sub-pixels, but no red quantum dots. Similarly, the blue sub-pixels can also get similar results by electro-testing, that is, only the peak appears at the position corresponding to the blue wavelength, but there is no peak at the position corresponding to the red wavelength, indicating that there are only blue quantum dots in the blue sub-pixels, but no red quantum dots. Therefore, it can be determined that the red quantum dot light emitting layer formed by the crosslinking agent TPE-4N₃ can be washed away in the unexposed area by the developing solution without remaining when the red quantum dot light emitting layer is developed by the developing solution.

Next, the inventors further explored the patterning of green quantum dots. The inventors prepared the green quantum dot light emitting layer with reference to the preparation method of the red quantum dot light emitting layer. Specifically, firstly, an electron transport layer solution is spin-coated on a substrate, wherein the material of the electron transport layer may be, for example, ZnO, and ZnO has a nanoparticle structure. For example, the electron transport layer may be formed by a sol-gel method. For example, the concentration of ZnO in the solution may be 75 mg/mL, the spin-coating speed may be 3000 rpm, and the heating temperature required in the sol-gel process may be about 300° C. Then, a mixed solution including crosslinking agent TPE-4N₃, green quantum dots with ligands on their surfaces, and chlorobenzene solvent is prepared. The ligand on the surface of green quantum dots is oleic acid. The concentration of green quantum dots in the mixed solution is 30 mg/mL, and the concentration of crosslinking agent in the mixed solution is 1 mg/mL. The mixed solution is spin-coated on the electron transport layer and cured to form a cured layer. Then, ultraviolet light was used for exposure, and the exposure time may be about 60 s, for example. Then, the exposed green quantum dot light emitting layer is developed under three different development conditions: (1) developing solution with 1% concentration for 30 s, (2) developing solution with 1% concentration for 60 s and (3) developing solution with 1.25% concentration for 30 s.

FIG. 32 shows green quantum dot patterns obtained under three different development conditions, in which FIG. 32(a) corresponds to development condition (1), FIG. 32(b) corresponds to development condition (2) and FIG. 32(c) corresponds to development condition (3). As can be seen from FIG. 32, the three kinds of green quantum dot light emitting layers have not formed the expected regular pattern shape in the exposed area, on the contrary, they are all disordered patterns, and the parts of the three kinds of green quantum dot light emitting layers in the unexposed area are basically not washed away by the developing solution.

The green quantum dot light emitting layer has the same ligands as the red quantum dot light emitting layer, and uses the same crosslinking agent, solvent, developing solution, etc. The only difference is that the luminescent colors of the quantum dots are different, but as shown in the figures, the patterning results of the two are absolutely different. The inventors believe that this may be due to the poor colloidal stability of green quantum dots, and the surface of electron transport layer ZnO may have defects. Since the volume of green quantum dots is usually smaller than the volume of red quantum dots, the green quantum dots are more likely to combine with defects on the surface of ZnO than red quantum dots, so it is more difficult to be washed away by developing solution, showing obvious residual phenomenon.

In order to solve the patterning problem of green quantum dots, the inventors proposed that it is necessary to explore new ligand materials and change the polarity of ligands on the surface of green quantum dots, so as to adjust the interaction between green quantum dot ligands and ZnO.

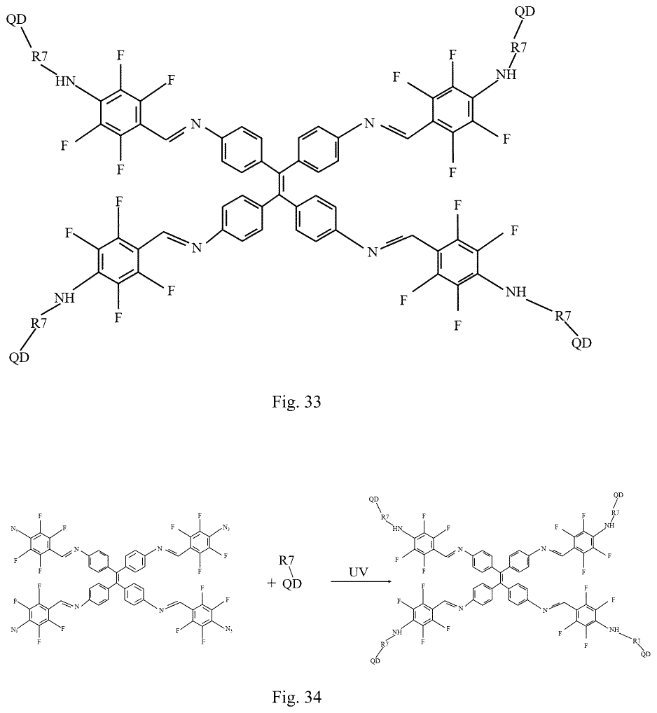

FIG. 33 shows a chemical formula of a quantum dot light emitting layer, the chemical formula is

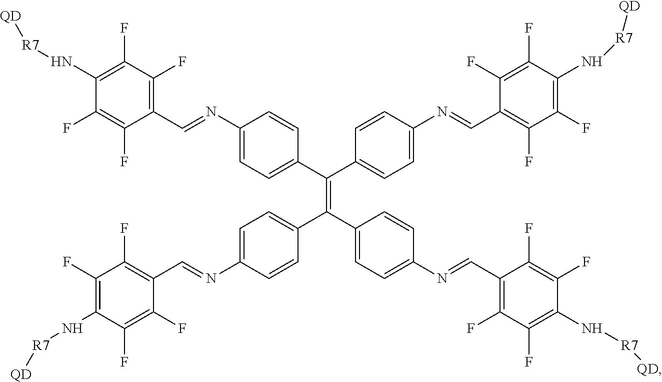

in which QD represents a quantum dot and R7 represents a ligand on the surface of the quantum dot. R7 is 2-mercaptoethyl Boc amine, and the chemical formula of 2-mercaptoethyl Boc amine is

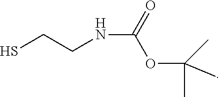

The quantum dot light emitting layer is a green quantum dot light emitting layer configured to emit green light.

FIG. 34 shows a synthesis route of the green quantum dot light emitting layer of FIG. 33, and the preparation process of the green quantum dot light emitting layer of FIG. 33 will be roughly described with reference to FIG. 34.

Firstly, a solution of the electron transport layer was spin-coated on the substrate to form the electron transport layer. The electron transport layer may be formed of any suitable material, for example, the material of the electron transport layer may be nano-particle type zinc oxide (ZnO), or metal-doped nano-zinc oxide, or Sol-Gel type zinc oxide (ZnO). For example, metal-doped nano-zinc oxide may include zinc oxide (ZnO) doped with magnesium (Mg), aluminum (Al), zirconium (Zr) or yttrium (Y). In some embodiments, the material of the electron transport layer is ZnO with nanoparticle structure, for example, ZnO with nanoparticle structure may be formed by sol-gel method. In some embodiments, the concentration of ZnO in the solution may be 75 mg/mL, the speed of spin coating the solution on the substrate may be 3000 rpm, and the heating temperature required in the sol-gel process may be about 300° C.

Then, a mixed solution including a crosslinking agent TPE-4N₃, green quantum dots with ligands, and chlorobenzene solvent was prepared. The ligand R7 on the surface of the green quantum dot QD is 2-mercaptoethyl Boc amine. In order to make the experimental results more universal, three kinds of mixed solutions with different concentrations were prepared. In a mixed solution, the concentration of green quantum dots QD-R7 is 30 mg/mL, and the concentration of crosslinking agent TPE-4N₃ is 1 mg/ml. In another mixed solution, the concentration of green quantum dots QD-R7 is 25 mg/mL, and the concentration of crosslinking agent TPE-4N₃ is 1 mg/mL. In another mixed solution, the concentration of green quantum dots QD-R7 is 20 mg/mL, and the concentration of crosslinking agent TPE-4N₃ is 1 mg/mL. Three kinds of mixed solutions were spin-coated on the electron transport layer and cured to form three kinds of cured layers. Then, the cured layer was exposed by ultraviolet light for about 60 s, for example, to form a crosslinked structure

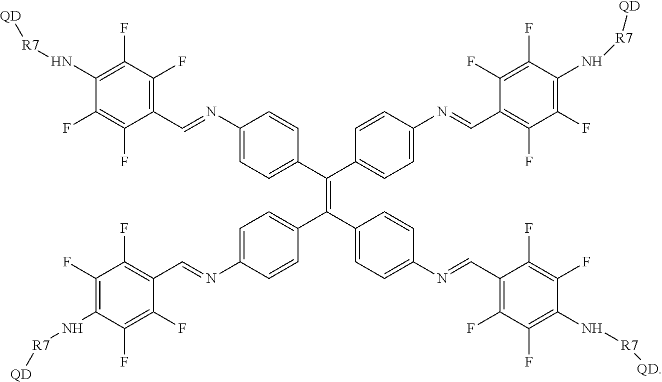

Then, the three kinds of crosslinked structures after exposure were developed by developing solution, and the development time may be about 30 s, for example.

It should be noted that the ligand 2-mercaptoethyl Boc amine includes a plurality of carbon-hydrogen bonds, and the azide group N₃ of the crosslinking agent TPE-4N₃ can undergo the nitrogen-hydrogen insertion reaction at any position of the carbon-hydrogen bonds of 2-mercaptoethyl Boc amine, and the insertion reaction position is not limited. Therefore, in the chemical formula

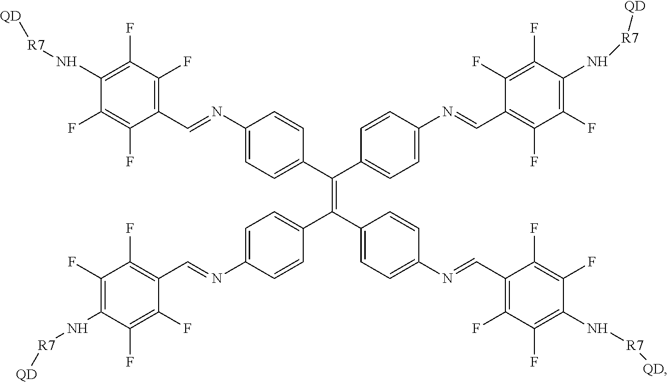

NH can be connected with any carbon atom of R7 (i.e. 2-mercaptoethyl Boc amine).

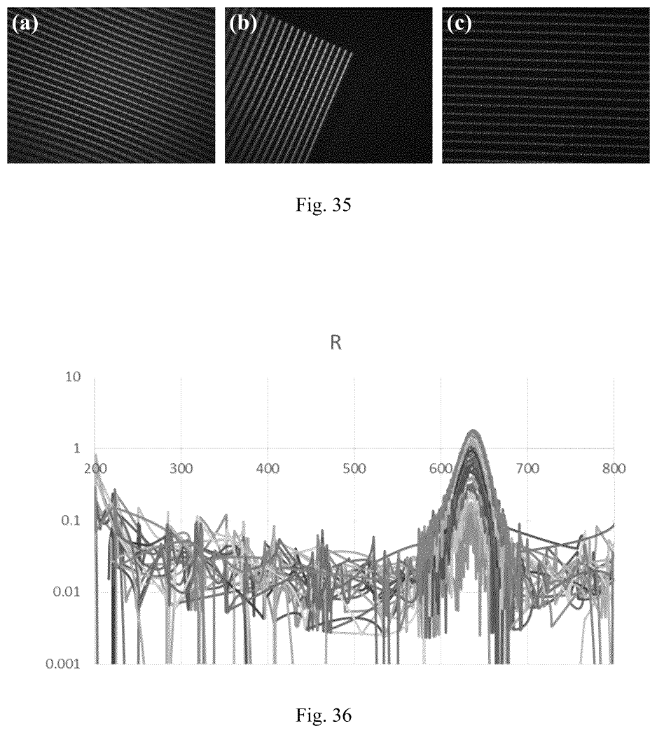

FIG. 35 shows the light-induced patterning of three kinds of green quantum dot light emitting layers obtained by using mixed solutions with three different concentrations. FIG. 35(a) corresponds to that the concentration of green quantum dots QD-R7 is 30 mg/mL, and the concentration of crosslinking agent TPE-4N₃ is 1 mg/ml; FIG. 35(b) corresponds to that the concentration of green quantum dots QD-R7 is 25 mg/mL, and the concentration of crosslinking agent TPE-4N₃ is 1 mg/mL; FIG. 35(c) corresponds to that the concentration of green quantum dots QD-R7 is 20 mg/mL, and the concentration of crosslinking agent TPE-4N₃ is 1 mg/mL. In FIGS. 35(a), 35(b) and 35(c), oblique or horizontal straight lines indicate the shapes of the patterned green quantum dot light emitting layers in the exposed areas in the lit state, and black areas indicate unexposed areas.

As can be seen from FIG. 35, the three kinds of green quantum dot light emitting layers all have good pattern shapes in the exposed areas, which are not damaged by the developing solution. And under the microscope, the parts of three kinds of green quantum dot light emitting layers in unexposed areas (such as adjacent red sub-pixel region and blue sub-pixel region) are all washed away by the developing solution without residue. This shows that using ligand 2-mercaptoethyl Boc amine instead of ligand oleic acid changes the polarity of ligands on the surface of green quantum dots, thus weakening the interaction between the ligands of green quantum dots and the electron transport layer ZnO, making the part of the light emitting layer of green quantum dots in the unexposed area easier to be washed away by the developing solution, thereby solving the residual problem and avoiding the phenomenon of color mixing.

The green quantum dot light emitting layer has high stability, and under the action of the developing solution, high-quality quantum dot patterns can be formed in exposed areas, and quantum dot residues in unexposed areas can be avoided, thus avoiding color mixing. The reason why the green quantum dot light emitting layer has high stability is that the green quantum dot light emitting layer is generated by crosslinking a crosslinking agent TPE-4N₃ and green quantum dots containing a 2-mercaptoethyl Boc amine ligands, and each crosslinking agent molecule TPE-4N₃ has four azide groups N₃. Since the azide groups N₃ have high photoinitiation efficiency and low exposure dose, and can generate nitrogen carbene free radicals under light radiation, the crosslinking agent TPE-4N₃ has high activity. When the crosslinking agent TPE-4N₃ and the green quantum dots containing 2-mercaptoethyl Boc amine ligands crosslink under light radiation, the crosslinking agent TPE-4N₃ can undergo the nitrogen-hydrogen insertion reaction with the carbon-hydrogen bonds of the 2-mercaptoethyl Boc amine ligands on the surface of the quantum dots to form a stable crosslinking structure, so that the formed green quantum dot light emitting layer has high stability and will not be destroyed by the developing solution, thus a high-quality green quantum dot pattern can be formed in the exposed area. Moreover, it further shows that the crosslinking agent TPE-4N₃ is an ideal crosslinking agent.

In order to further verify that there is no green quantum dot residue in the unexposed area, red quantum dot patterns and blue quantum dot patterns were further prepared on the basis of any of the above three green quantum dot patterns, and then the hole transport layer material and the electrode material were evaporated, and the devices were packaged to form quantum dot light emitting devices.

FIG. 36 shows an electrospectrogram of red sub-pixels of a quantum dot light emitting device. When the red sub-pixels are tested by electro-testing, with the gradual increase of voltage (for example, from 4V to 8V), it is found that the electrospectrogram only has a peak at the position corresponding to the red wavelength, but there is no peak at the position corresponding to the green wavelength. This shows that there are only red quantum dots in the red sub-pixel and no green quantum dots. Similarly, if the blue sub-pixels are electrically tested, similar results can be obtained, that is, only the peak appears at the position corresponding to the blue wavelength, but there is no peak at the position corresponding to the green wavelength, indicating that there are only blue quantum dots in the blue sub-pixel and no green quantum dots. Therefore, it can be determined that the green quantum dot light emitting layer formed by the crosslinking agent TPE-4N₃ and quantum dots containing 2-mercaptoethyl Boc amine ligands can be washed away by the developing solution and will not remain when the green quantum dot light emitting layer is developed by the developing solution. This shows that the residual problem of green quantum dots has been solved.

Next, the inventors further explored the patterning of blue quantum dots. The inventors prepared the blue quantum dot light emitting layer with reference to the preparation method of the red quantum dot light emitting layer. The chemical formula of the blue quantum dot light emitting layer

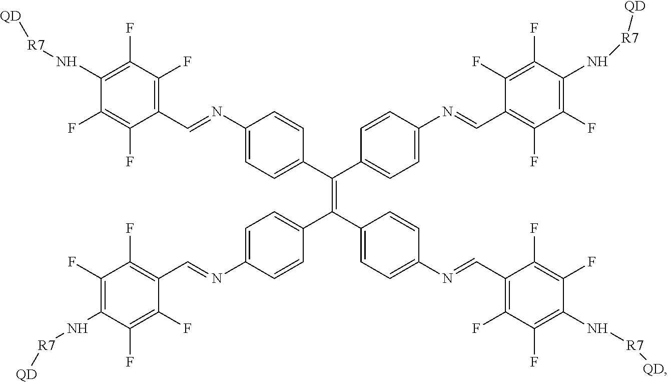

in which QD represents quantum dot and R7 represents ligand on the surface of quantum dot. R7 is oleic acid, and its chemical formula is

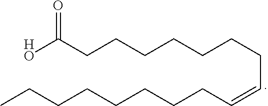 [SMILES: CCCCCCCC/C=C\CCCCCCCC(=O)O]

Specifically, firstly, a solution of the electron transport layer was spin-coated on the substrate to form the electron transport layer. The electron transport layer may be formed of any suitable material, for example, the material of the electron transport layer may be nano-particle type zinc oxide (ZnO), or metal-doped nano-zinc oxide, or Sol-Gel type zinc oxide (ZnO). For example, metal-doped nano-zinc oxide may include zinc oxide (ZnO) doped with magnesium (Mg), aluminum (Al), zirconium (Zr) or yttrium (Y). In some embodiments, the material of the electron transport layer is ZnO with nanoparticle structure, for example, ZnO with nanoparticle structure may be formed by sol-gel method. In some embodiments, the concentration of ZnO in the solution may be 75 mg/mL, the speed of spin coating the solution on the substrate may be 3000 rpm, and the heating temperature required in the sol-gel process may be about 300° C.

Then, a mixed solution including a crosslinking agent TPE-4N₃, blue quantum dots with ligands, and chlorobenzene solvent was prepared. The ligand on the surface of blue quantum dots is oleic acid. The concentration of blue quantum dots in the mixed solution is 30 mg/mL, and the concentration of crosslinking agent TPE-4N₃ in the mixed solution is 1 mg/mL. The mixed solution was spin-coated on the electron transport layer and cured to form a cured layer. Then, ultraviolet light was used for exposure, and the exposure time may be about 30 s, for example. Then, the exposed blue quantum dot light emitting layer was developed under two different development conditions: (a) developing solution with 1% concentration for 30 s, and (b) developing solution with 0.75% concentration for 30 s. The four azide groups N₃ of crosslinking agent TPE-4N₃ can generate nitrogen carbene free radicals under ultraviolet irradiation, which has high activity and can undergo nitrogen-hydrogen insertion reaction with the carbon-hydrogen bond of oleic acid ligand R7 on the surface of quantum dots. After the insertion reaction of nitrogen and hydrogen, the azide group N₃ becomes NH, which is connected with the ligand R7, thus forming the blue quantum dot light emitting layer with crosslinking structure. As mentioned above, NH can be attached to any carbon atom of oleic acid ligand R7.

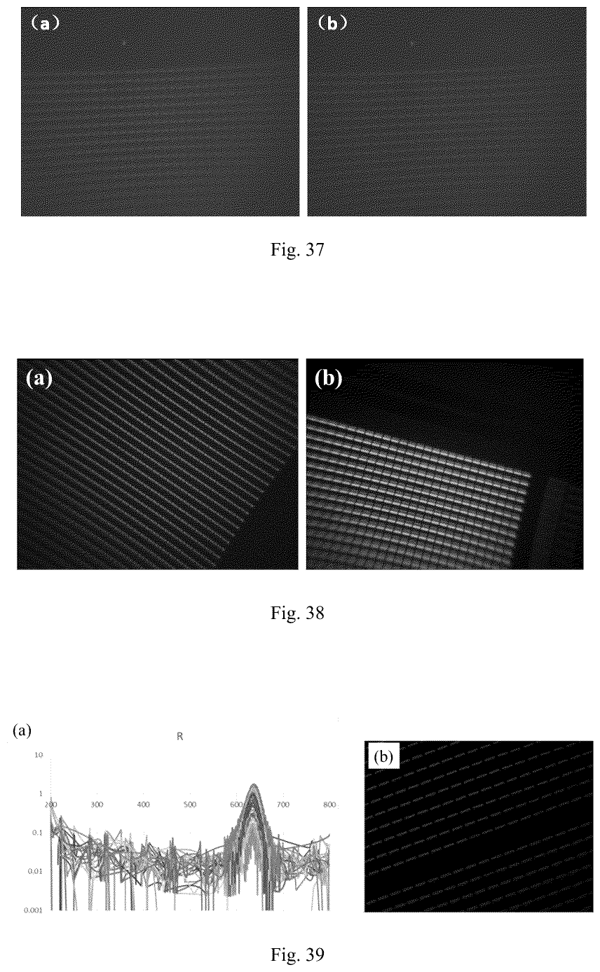

FIG. 37 shows photos of light-induced patterning of blue quantum dot light emitting layers obtained under two different development conditions, in which FIG. 37(a) corresponds to the above development condition (a) and FIG. 37(b) corresponds to the above development condition (b). The straight line in the photo represents the shape of the patterned blue quantum dot light emitting layer in the exposed area in the lit state, and the black area represents the unexposed area. As can be seen from FIG. 37, both blue quantum dot light emitting layers have good pattern shapes in the exposed areas, which are not damaged by the developing solution, and the parts of the two blue quantum dot light emitting layers in the unexposed areas are washed away by the developing solution, and no blue quantum dots remain. This shows that the blue quantum dot light emitting layer has high stability, and under the action of developing solution, it can not only form high-quality blue quantum dot patterns in exposed areas, but also avoid blue quantum dot residues in unexposed areas and avoid color mixing.

The reason why the blue quantum dot light emitting layer has high stability is that the blue quantum dot light emitting layer is generated by crosslinking the crosslinking agent TPE-4N₃ and blue quantum dots containing oleic acid ligands, and each crosslinking agent molecule TPE-4N₃ has four azide groups N₃. Since the azide groups N₃ have high photoinitiation efficiency and low exposure dose, and can generate nitrogen carbene free radicals under light radiation, the crosslinking agent TPE-4N₃ has high activity. When the crosslinking agent TPE-4N₃ and the blue quantum dots containing oleic acid ligands crosslink under light radiation, the crosslinking agent TPE-4N₃ can undergo the nitrogen-hydrogen insertion reaction with the carbon-hydrogen bonds of the oleic acid ligands on the surface of the quantum dots to form a stable crosslinking structure, so that the formed blue quantum dot light emitting layer has high stability and will not be destroyed by the developing solution, thus a high-quality blue quantum dot pattern can be formed in the exposed area. Moreover, it further shows that the crosslinking agent TPE-4N₃ is an ideal crosslinking agent.

The inventors further carried out the experiment of red and green quantum dots, exploring the residue of green quantum dots on the patterned red quantum dot light emitting layer, and the performance of the red quantum dot light-emitting device and the green quantum dot light-emitting device after the lithography process.

Firstly, a solution of the electron transport layer was spin-coated on the substrate to form the electron transport layer. The electron transport layer may be formed of any suitable material, for example, the material of the electron transport layer may be nano-particle type zinc oxide (ZnO), or metal-doped nano-zinc oxide, or Sol-Gel type zinc oxide (ZnO). For example, metal-doped nano-zinc oxide may include zinc oxide (ZnO) doped with magnesium (Mg), aluminum (Al), zirconium (Zr) or yttrium (Y). In some embodiments, the material of the electron transport layer is ZnO with nanoparticle structure, for example, ZnO with nanoparticle structure can be formed by sol-gel method. In some embodiments, the concentration of ZnO in the solution may be 75 mg/mL, the speed of spin coating the solution on the substrate may be 3000 rpm, and the heating temperature required in the sol-gel process may be about 300° C.

Then, a first mixed solution including a crosslinking agent TPE-4N₃, red quantum dots with ligands, and chlorobenzene solvent was prepared. The ligand on the surface of red quantum dots is oleic acid. The concentration of red quantum dots in the first mixed solution is 30 mg/mL, and the concentration of crosslinking agent TPE-4N₃ in the first mixed solution is 1 mg/mL. The first mixed solution was spin-coated on the electron transport layer and cured to form a cured layer. Then, ultraviolet light was used for exposure, and the exposure time may be about 60 s, for example. Then, a developing solution with a concentration of 1% was used to develop for 30 s to form a patterned red quantum dot monolayer pattern, as shown in FIG. 38(a).

Then, a second mixed solution including crosslinking agent TPE-4N₃, green quantum dots with ligands, and chlorobenzene solvent was spin-coated, and the ligand on the surface of the green quantum dots is 2-mercaptoethyl Boc amine. The concentration of green quantum dots in the second mixed solution is 20 mg/mL, and the concentration of crosslinking agent TPE-4N₃ in the second mixed solution is 1 mg/mL. Then, ultraviolet light was used for exposure, the exposure time may be about 60 s, for example. Then, a developing solution with a concentration of 1% was used to develop for 30 s, and the patterns of red quantum dots and green quantum dots were obtained, as shown in FIG. 38(b). Then, the hole transport layer material and electrode material were evaporated, and the device was packaged to form a light emitting device including red quantum dots and green quantum dots.

FIG. 39(a) shows an electrospectrogram of the red sub-pixels of the light emitting device corresponding to FIG. 38, and FIG. 39(b) shows a photo of the light-induced patterning of the red sub-pixels of the light emitting device corresponding to FIG. 38. As shown in FIG. 39(a), when the red sub-pixels are tested by electro-testing, with the gradual increase of voltage (for example, from 4V to 8V), it is found that the peak appeared only at the position corresponding to the red wavelength, but there is no peak at the position corresponding to the green wavelength. This shows that there are only red quantum dots in the red sub-pixel, but there are no green quantum dots, and the green quantum dots have no effect on the patterned red quantum dot film during the lithography process. As can be seen from FIG. 39(b), the quantum dot film layer of the red sub-pixel has a good pattern shape, and its morphology is complete and undamaged.

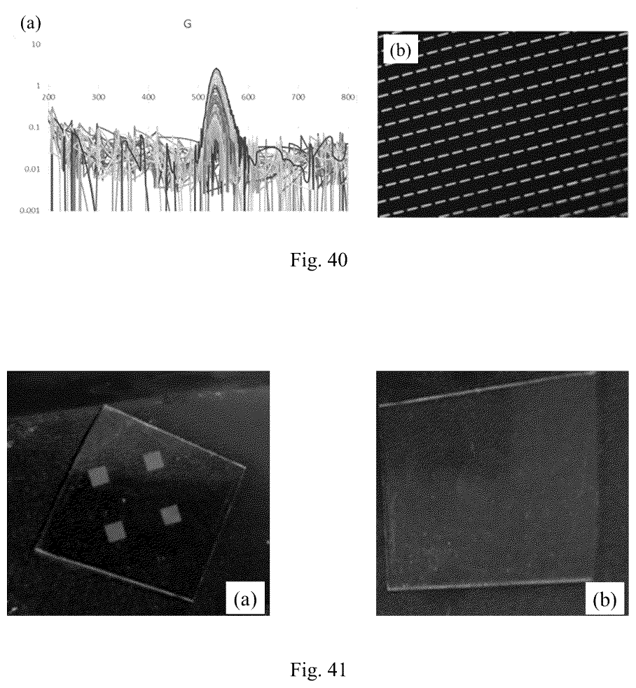

FIG. 40(a) shows an electrospectrogram of the green sub-pixels of the light emitting device corresponding to FIG. 38, and FIG. 40(b) shows a photo of the light-induced patterning of the green sub-pixel of the light emitting device corresponding to FIG. 38. As shown in FIG. 40(a), when the green sub-pixels are tested by electro-testing, with the gradual increase of voltage (for example, from 4V to 8V), it is found that the peak appeared only at the position corresponding to the green wavelength, but not at the position corresponding to the red wavelength. This shows that there are only green quantum dots in the green sub-pixel, but no red quantum dots remain. As can be seen from FIG. 40(b), the quantum dot light emitting layer of the green sub-pixel has a good pattern shape, and the morphology is complete and undamaged.

With reference to FIG. 39 and FIG. 40, in a light emitting device including red quantum dots and green quantum dots, the quantum dot light emitting layer includes a plurality of first quantum dot patterns, a plurality of second quantum dot patterns, a plurality of first sub-pixel regions, and a plurality of second sub-pixel regions, wherein the first quantum dot patterns may be red quantum dot patterns for emitting red light, and the second quantum dot patterns may be green quantum dot patterns for emitting green light. The first quantum dot patterns are only in the first sub-pixel regions but not in the second sub-pixel regions, and the second quantum dot patterns are only in the second sub-pixel regions but not in the first sub-pixel regions. In short, the quantum dot light emitting layer formed by using the crosslinking agent provided by the embodiments of the present disclosure has high-quality quantum dot patterns, and each sub-pixel has no color mixing phenomenon, so that the color gamut of the quantum dot electroluminescent device can be improved.

In some embodiments, in an electroluminescent device including red quantum dots, green quantum dots and blue quantum dots, the quantum dot light emitting layer includes a plurality of first quantum dot patterns, a plurality of second quantum dot patterns, a plurality of third quantum dot patterns, a plurality of first sub-pixel regions, a plurality of second sub-pixel regions and a plurality of third sub-pixel regions. The first quantum dot patterns may be red quantum dot patterns for emitting red light, the second quantum dot patterns may be green quantum dot patterns for emitting green light, the third quantum dot patterns may be blue quantum dot patterns for emitting blue light, the first sub-pixel regions may be red sub-pixel regions, the second sub-pixel regions may be green sub-pixel regions, and the third sub-pixel regions may be blue sub-pixel regions. The first quantum dot patterns are only in the first sub-pixel region but not in the second sub-pixel regions and the third sub-pixel regions, the second quantum dot patterns are only in the second sub-pixel regions but not in the first sub-pixel regions and the third sub-pixel regions. In short, the quantum dot light emitting layer formed by using the crosslinking agent provided by the embodiments of the present disclosure has high-quality quantum dot patterns, and each sub-pixel has no color mixing phenomenon, so that the color gamut of the quantum dot electroluminescent device can be improved.

As a comparative example, FIG. 41 shows a photo of a patterned red quantum dot light emitting layer, and the crosslinking agent used is

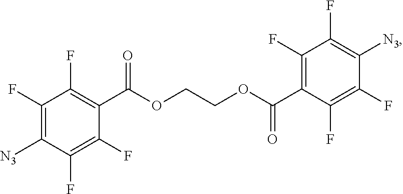 [SMILES: [N-]=[N+]=Nc1c(F)c(F)c(C(=O)OCCOC(=O)c2c(F)c(F)c(N=[N+]=[N-])c(F)c2F)c(F)c1F]

each crosslinking agent molecule has only two azide groups N₃. The preparation method of the red quantum dot light emitting layer is described below.

Firstly, a solution of the electron transport layer was spin-coated on a substrate to form an electron transport layer.

Then, a mixed solution including a crosslinking agent, red quantum dots with ligands on the surface, and a solvent was prepared. The crosslinking agent is

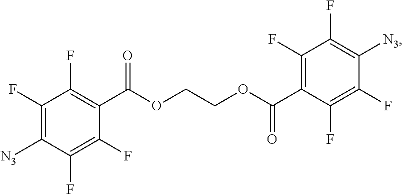 [SMILES: [N-]=[N+]=Nc1c(F)c(F)c(C(=O)OCCOC(=O)c2c(F)c(F)c(N=[N+]=[N-])c(F)c2F)c(F)c1F]

the ligand on the surface of red quantum dots is oleic acid, the solvent is chlorobenzene. The concentration of red quantum dots in the mixed solution is 20 mg/mL, and the concentration of crosslinking agent in the mixed solution is 1 mg/mL. Then, the mixed solution was spin-coated on the electron transport layer and cured to form a cured layer. Then, ultraviolet light was used for exposure, and the exposure time may be about 60 s, for example. Then, the exposed red quantum dot light emitting layer was developed by the developing solution. Ideally, the obtained red quantum dot pattern should be as shown in FIG. 41(a), but in reality, the obtained red quantum dot pattern is as shown in FIG. 41(b) after being developed by the developing solution, and the red quantum dot pattern can't withstand the immersion of the developing solution and has been basically washed away by the developing solution.

The phenomenon shown in FIG. 41(b) occurs because the crosslinking agent

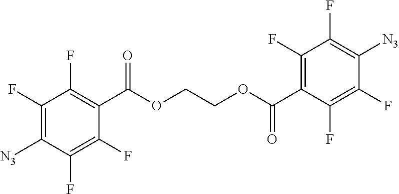 [SMILES: [N-]=[N+]=Nc1c(F)c(F)c(C(=O)OCCOC(=O)c2c(F)c(F)c(N=[N+]=[N-])c(F)c2F)c(F)c1F]

only has two azide groups N₃, and the number of azide groups N₃ is too small. In the process of crosslinking with quantum dots, the crosslinking agent cannot provide sufficient photoinitiation efficiency, resulting in insufficient degree of crosslinking between quantum dots and the crosslinking agent in the exposed area, which in turn leads to the poor stability of the formed red quantum dot light emitting layer, which cannot withstand the immersion of developing solution and thus cannot form the expected pattern shape.

As described above, in the crosslinking agents provided by various embodiments of the present disclosure, the number of azide groups N₃ in each crosslinking agent molecule is greater than or equal to 3, for example, the number of azide groups N₃ in each crosslinking agent molecule may be 3, 4, 5, 6, 8 or even more than 100, and the number of azide groups N₃ can ensure that the crosslinking agent has high photoinitiation efficiency. The quantum dot light emitting layer formed by the crosslinking agent and quantum dots provided by various embodiments of the present disclosure has high stability and can withstand the immersion of developing solution, thus forming the expected pattern shape. The high stability of the quantum dot light emitting layers provided by the embodiments of the disclosure have been verified by experiments in the previous embodiments.

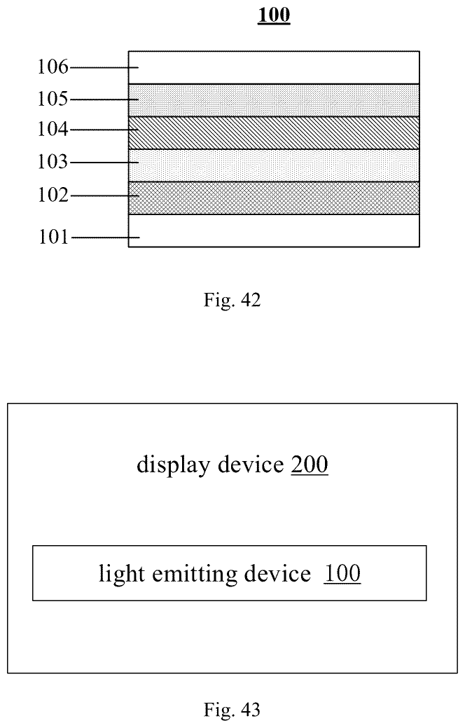

FIG. 42 shows a schematic structural diagram of a light emitting device 100. As shown in FIG. 42, the light emitting device 100 includes a first electrode layer 101; an electron transport layer 102 on the first electrode layer 101; a quantum dot light emitting layer 103 on a side of the electron transport layer 102 away from the first electrode layer 101, which may be the quantum dot light emitting layer described in any of the previous embodiments; a hole transport layer 104 on a side of the quantum dot light emitting layer 103 away from the first electrode layer 101; and a second electrode layer 106 on a side of the hole transport layer 104 away from the first electrode layer 101.

The light emitting device 100 is an electroluminescent device, and its light emitting principle is that the quantum dot light emitting layer 103 is sandwiched between the first electrode layer 101 and the second electrode layer 106, under the action of an electric field, the first electrode layer 101 and the second electrode layer 106 generate electrons and holes respectively, and the electrons and holes are transmitted to the quantum dot light emitting layer 103, and are recombined into excitons in the quantum dot light emitting layer 103 to generate energy level transitions, thereby emitting light. The light emitting device 100 has the advantages of high color purity, high contrast and high stability.

In some embodiments, the light emitting device 100 may further include a hole injection layer 105 between the hole transport layer 104 and the second electrode layer 106.

In some embodiments, the first electrode layer 101 is a cathode and the second electrode layer 106 is an anode. In this case, the light emitting device 100 has an inverted structure. The first electrode layer 101 and the second electrode layer 106 may be formed of various suitable materials, which are not limited by the embodiments of the present disclosure. Illustratively, the material of the first electrode layer 101 may be ITO, and the material of the second electrode layer 106 may be Al.

In some alternative embodiments, the light emitting device 100 may have an upright structure. In this case, the lamination relationship of each film layer of the light emitting device 100 is as follows: an anode, a hole injection layer on the anode; a hole transport layer on a side of the hole injection layer away from the anode; a quantum dot light emitting layer on a side of the hole transport layer away from the anode; an electron transport layer on a side of the quantum dot light emitting layer away from the anode; and a cathode on a side of the electron transport layer away from the anode.

The light emitting device 100 may be a top emission type or a bottom emission type.

The electron transport layer 102 may be formed of any suitable material. For example, the material of the electron transport layer 102 may be nano-particle type zinc oxide (ZnO), metal-doped nano-zinc oxide, or Sol-Gel type zinc oxide (ZnO). For example, metal-doped nano-zinc oxide includes zinc oxide (ZnO) doped with magnesium (Mg), aluminum (Al), zirconium (Zr) or yttrium (Y). In some embodiments, the material of the electron transport layer 102 is ZnO with a nanoparticle structure, for example, ZnO with a nanoparticle structure may be formed by a sol-gel method.

The hole transport layer 104 may be formed of any suitable material, including but not limited to carrier transport materials containing carbazole groups, carbazole derivative groups, triphenylamine groups and triphenylamine derivative groups. In some embodiments, the material of the hole transport layer 104 may be any one of 1,2,4,5-tetra (trifluoromethyl) benzene (TFB), polyvinyl carbazole (PVK) and poly [bis(4-phenyl) (4-butylphenyl) amine] (Poly-TPD).

For the technical effect of the light emitting device 100, reference can be made to the technical effect of the quantum dot light emitting layer described in the previous embodiments, and for the sake of brevity, the description will not be repeated here.

FIG. 43 shows a block diagram of the display device 200. The display device 200 includes a plurality of sub-pixels, and each sub-pixel is provided with a light emitting device 100. At least two of the plurality of light emitting devices 100 emit light of different colors. For example, some of the plurality of light emitting devices 100 include red quantum dot light emitting layers and are used to emit red light; some of the plurality of light emitting devices 100 include green quantum dot light emitting layers and are used to emit green light; some of the plurality of light emitting devices 100 include blue quantum dot light emitting layer and are used to emit blue light, thereby enabling the display device 200 to realize full-color display.

Of course, the display device 200 also includes other components not shown, for example, it may include a driving circuit for providing an electrical signal to the light emitting device 100 to drive the light emitting device 100 to emit light. The display device 200 may also include a circuit board and/or an Integrate Circuit (IC).

The display device 200 may be a mobile phone, a tablet computer, a television, a display, a notebook computer, a digital photo frame, a navigator and other products or components that display based on quantum dots.

The display device 200 can have basically the same technical effect as the light emitting device 100 described in the previous embodiment, so for the sake of brevity, the description will not be repeated here.

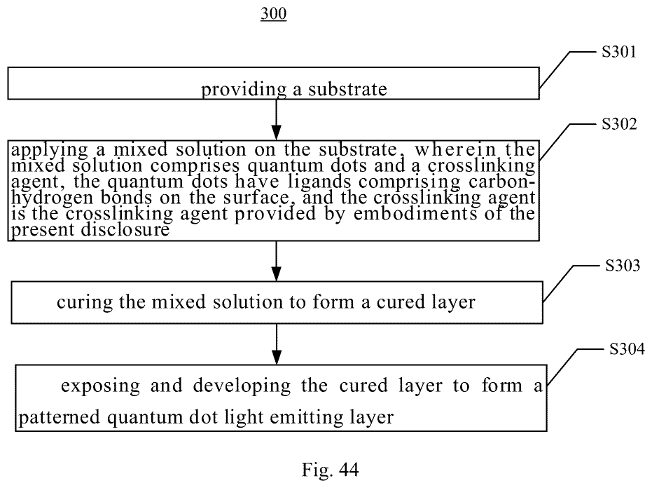

FIG. 44 shows a flowchart of a method 300 for preparing a patterned quantum dot light emitting layer. As shown in FIG. 44, the method 300 includes the following steps:
S301: providing a substrate.

The substrate may be an inorganic material, an organic material, a silicon wafer or a composite material layer, etc. the inorganic material may be glass, metal, for example; the organic material may be polycarbonate, polymethylmethacrylate, polyethylene terephthalate, polyethylene naphthalate, polyamide, polyethersulfone, or combinations thereof, for example.
S302: applying a mixed solution on the substrate, wherein the mixed solution comprises quantum dots and a crosslinking agent, wherein the quantum dots comprise quantum dot bodies and ligands coordinated on the quantum dot bodies, the ligands comprise carbon-hydrogen bonds, and the crosslinking agent is the crosslinking agent described in any of the previous embodiments; In some embodiments, the concentration of quantum dots in the mixed solution may be about 20~35 mg/mL, such as 20 mg/mL, 25 mg/mL, 30 mg/mL, 35 mg/mL. The concentration of crosslinking agent in the mixed solution may be about 0.1~2.0 mg/mL, such as 0.1 mg/mL, 0.5 mg/mL, 1.0 mg/mL, 1.5 mg/mL, 2.0 mg/mL.
S303: curing the mixed solution to form a cured layer.
S304: exposing and developing the cured layer to form a patterned quantum dot light emitting layer.

The crosslinking agent has the general formula R—(R1)ₙ, wherein R includes at least three reaction sites connected with R1. The group R may be

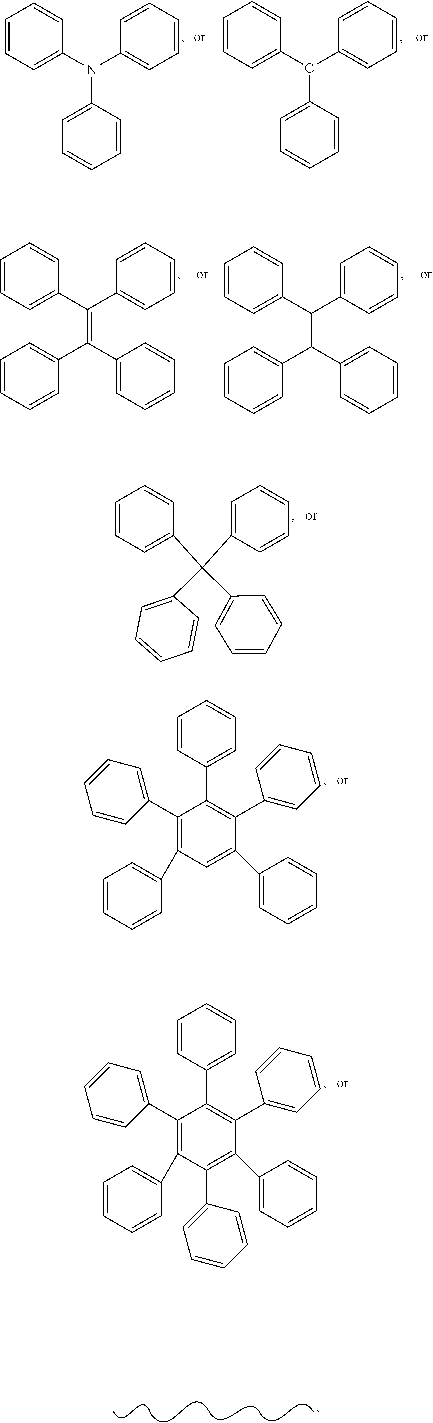

or other suitable groups.

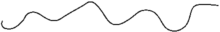

represents a polymer chain, and the functional group of the polymer chain includes at least one of carboxyl group, hydroxyl group and aldehyde group. The chemical formula of the group R1 is

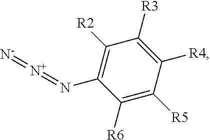

wherein any one of R2~R6 is a conjugated group or a non-conjugated group, which is connected with the reaction site on R, and the remaining groups in R2~R6 except the group connected with the reaction site on R may be the same or different from each other and are respectively selected from fluorine atoms or hydrogen atoms. In the general formula R—(R1)ₙ, n is a positive integer greater than or equal to 3, and the value of n is equal to the number of reaction sites on R.

In some embodiments, the para group R4 of the azide group N3 is connected with the reaction site on R. In this case, the group R4 is a conjugated group or a non-conjugated group, and the remaining groups R2, R3, R5 and R6 are the same or different from each other and may be fluorine atoms or hydrogen atoms respectively. In some alternative embodiments, the ortho group R2 (or R6) of the azide group N3 may be connected with the reaction site on R. In this case, the group R2 (or R6) is a conjugated group or a non-conjugated group, and the remaining groups R3, R4, R5, R6 (or R2, R3, R4, R5) are the same or different from each other and may be fluorine atoms or hydrogen atoms respectively. In some alternative embodiments, the meta group R3 (or R5) of the azide group N3 may be connected with the reaction site on R. In this case, the group R3 (or R5) is a conjugated group or a non-conjugated group, and the remaining groups R2, R4, R5, R6 (or R2, R3, R4, R6) are the same or different from each other and may be fluorine atoms or hydrogen atoms respectively.

In some embodiments, when R is

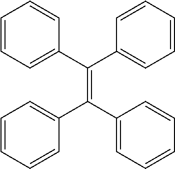 [SMILES: c1ccc(C(=C(c2ccccc2)c2ccccc2)c2ccccc2)cc1]

### and R1 is

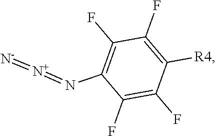

n in the general formula R—(R1)ₙ is equal to 4, and the chemical formula of the crosslinking agent may be expressed as

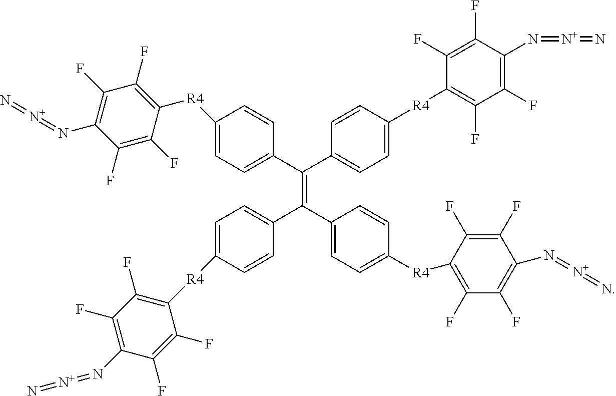

In this chemical formula, the middle mother nucleus R is selected as tetraethylene, which includes four reaction sites, so it can be connected with four groups R1, that is, the crosslinking agent includes four azide groups N₃. Since azide group N₃ has high photoinitiation efficiency, the crosslinking agent with tetrasubstituted azide group N₃ has good crosslinking degree, and the stability of quantum dot light emitting layer can be improved by using this crosslinking agent.

The selection of the group R4 of the crosslinking agent mainly considers the solubility, the source of raw materials and the difficulty of post-treatment. In terms of solubility, according to the principle of similar miscibility between molecules, the crosslinking agent is guaranteed to have good compatibility with quantum dots. For example, when the solvent of quantum dots contains ester groups, the group R4 of the crosslinking agent can be selected as the ester group. When the solvent of quantum dots contains amide groups, the group R4 of the crosslinking agent can be selected as amide group. When the solvent of quantum dots contains carbon-nitrogen double bonds, the group R4 of the crosslinking agent can be selected as carbon-nitrogen double bonds. When the solvent of quantum dots contains carbon-carbon double bonds, the group R4 of the crosslinking agent can be selected as carbon-carbon double bonds. When the solvent of quantum dots contains ether group, the group R4 of the crosslinking agent can be selected as ether group.

In some embodiments, the chemical formula of the crosslinking agent may be

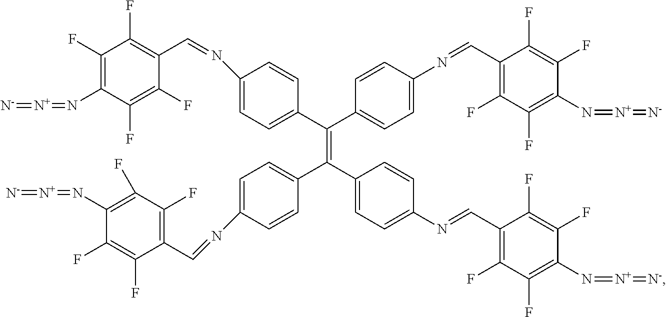

or may be

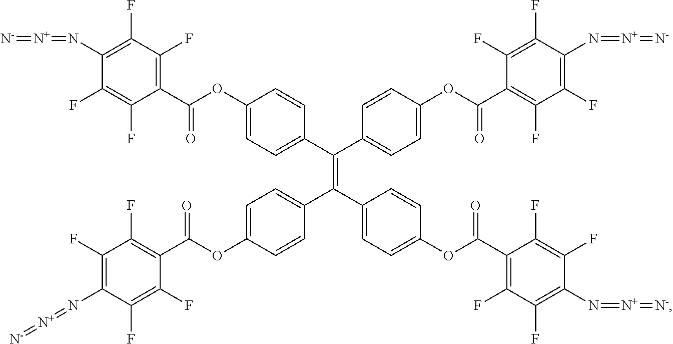

or may be

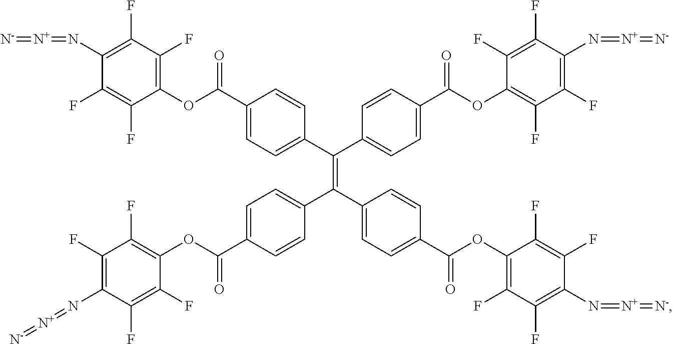

or may be

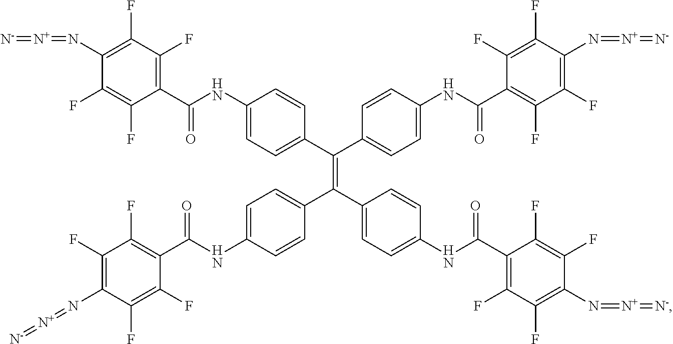

or may be

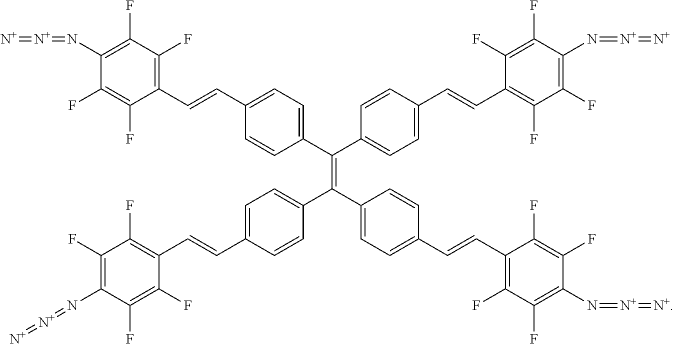

In some embodiments, step S304 may further include the following sub-steps: exposing the cured layer by using a mask plate, allowing ultraviolet light to penetrate through the mask plate to expose the cured layer, and performing crosslinking reaction between the crosslinking agent and ligands on the surface of the quantum dots under ultraviolet light irradiation; and developing a crosslinked cured layer by using a developing solution, wherein a part of the cured layer in the non-exposed area is dissolved by the developing solution, and a part of the cured layer in the exposed area is not dissolved by the developing solution, so as to form the patterned quantum dot light emitting layer.

The quantum dots provided by the embodiments of the present disclosure are negative quantum dot materials. Specifically, in the preparation process, the mixed solution including quantum dots and the crosslinking agent is cured to form a cured layer, and under light radiation, the chemical composition of the exposed part of the cured layer changes, while the chemical composition of the unexposed part does not change. The unexposed part of the cured layer can be subsequently dissolved in a specific developing solution, while the exposed part is not dissolved by the developing solution, thus forming a patterned quantum dot light emitting layer. Therefore, during the preparation, the quantum dot film with photosensitive characteristics can be directly exposed and developed to obtain a patterned quantum dot light emitting layer without using photoresist and etching the quantum dot light emitting layer.

In the method 100, since the group R in the crosslinking agent has at least three reaction sites that can be connected with the group R1, each group R can be connected with at least three groups R1, and each R1 includes at least one azide group N₃, so the crosslinking agent includes at least three azide groups N₃. Since the azide group N₃ has high photoinitiation efficiency and low exposure dose, and under the ultraviolet exposure condition, azide group N₃ in the crosslinking agent can generate nitrogen carbene free radicals with high activity, therefore, under the ultraviolet exposure condition, the crosslinking agent can undergo a nitrogen-hydrogen insertion reaction with the carbon-hydrogen bond of the quantum dot ligand to form a quantum dot light emitting layer with a crosslinked structure. The formed quantum dot light emitting layer has high stability, and under the action of developing solution, the part of the quantum dot light emitting layer in the exposure area will not be damaged by developing solution, and the expected pattern shape can be well formed. At the same time, the part of the quantum dot light emitting layer in the unexposed area can be washed away by the developing solution without residue, thus avoiding the color mixing phenomenon caused by the residue of quantum dots. In this way, the problem of quantum dot residue is solved, and high-quality quantum dot patterns can be obtained.

It will be understood that, the term “at least one of A, B, and C” has the same meaning as the term” at least one of A, B, or C” and both include the following combinations of A, B, and C: only A, only B, only C, combinations of A and B, combinations of A and C, combinations of B and C, and combinations of A, B, and C.

The term “A and/or B” includes the following three combinations: only A, only B, and a combination of A and B.

As used herein, “about”, “roughly” or “approximately” includes the stated value and an average value within an acceptable deviation range of a specific value, which is determined by a person of ordinary skill in the art in consideration of the measurement in question and the error (i.e., the limitations of the measurement system) associated with the measurement of a specific quantity.

It will be understood that although the terms first, second, third, etc. may be used herein to describe various elements, compositions, regions, layers and/or parts, these elements, compositions, regions, layers and/or parts should not be limited by these terms. These terms are only used to distinguish an element, composition, region, layer or part from another element, composition, region, layer or part. Thus, a first element, composition, region, layer or part discussed above could be termed a second element, composition, region, layer or part without departing from the teachings of the present disclosure.

Spatially relative terms such as “row”, “column”, “below”, “above”, “left”, “right”, etc. may be used herein for ease of description to describe factors such as the relationship of an element or feature to another element(s) or feature(s) illustrated in the figures. It will be understood that these spatially relative terms are intended to encompass different orientations of the device in use or operation in addition to the orientation depicted in the figures. For example, if the device in the figures is turned over, elements described as “below” other elements or features would then be oriented “above” other elements or features. Thus, the exemplary term “below” can encompass both an orientation of above and below. The device may be otherwise oriented (rotated 90 degrees or at other orientations) and the spatially relative descriptors used herein are interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to limit the present disclosure. As used herein, the singular forms “a,” “an,” and “the” are intended to comprise the plural forms as well, unless the context clearly dictates otherwise. It will be further understood that the terms “comprise” and/or “include” when used in this specification designate the presence of stated features, integers, steps, operations, elements and/or parts, but do not exclude the presence or addition of one or more other features, integers, steps, operations, elements, compositions and/or groups thereof. As used herein, the term “and/or” comprises any and all combinations of one or more of the associated listed items. In the description of this specification, description with reference to the terms “an embodiment,” “another embodiment,” etc. means that a particular feature, structure, material, or characteristic described in connection with the embodiment is included in at least one embodiment of the present disclosure. In this specification, schematic representations of the above terms are not necessarily directed to the same embodiment or example. Furthermore, the particular features, structures, materials or characteristics described may be combined in any suitable manner in any one or more embodiments or examples. Furthermore, those skilled in the art may combine the different embodiments or examples as well as the features of the different embodiments or examples described in this specification without conflicting each other.

It will be understood that when an element or layer is referred to as being “on”, “connected to”, “coupled to”, or “adjacent to” another element or layer, it may be directly on, directly connected to, directly coupled to, or directly adjacent to another element or layer, or intervening elements or layers may be present. In contrast, when an element is referred to as being “directly on”, “directly connected to”, “directly coupled to”, “directly adjacent to” another element or layer, with no intervening elements or layers present. However, in no case should “on” or “directly on” be interpreted as requiring a layer to completely cover the layer below.

Embodiments of the disclosure are described herein with reference to schematic illustrations (and intermediate structures) of idealized embodiments of the disclosure. As such, variations to the shapes of the illustrations are to be expected, e.g., as a result of manufacturing techniques and/or tolerances. Accordingly, embodiments of the present disclosure should not be construed as limited to the particular shapes of the regions illustrated herein, but are to comprise deviations in shapes due, for example, to manufacturing. Thus, the regions illustrated in the figures are schematic in nature and their shapes are not intended to illustrate the actual shape of a region of a device and are not intended to limit the scope of the present disclosure.

Unless otherwise defined, all terms (comprising technical and scientific terms) used herein have the same meaning as commonly understood by one of ordinary skill in the art to which this disclosure belongs. It will be further understood that terms such as those defined in commonly used dictionaries should be construed to have meanings consistent with their meanings in the relevant art and/or the context of this specification, and will not be idealized or overly interpreted in a formal sense, unless expressly defined as such herein.

As will be appreciated by those skilled in the art, although the steps of the methods of the present disclosure are depicted in a particular order in the figures, this does not require or imply that the steps must be performed in that particular order, unless the context clearly dictates otherwise. Additionally or alternatively, multiple steps may be combined into one step for execution, and/or one step may be decomposed into multiple steps for execution. Furthermore, other method steps may be inserted between the steps. The inserted steps may represent such as improvements of a method described herein, or may be unrelated to the method. Also, a given step may not be fully complete before the next step starts.

The above descriptions are merely specific embodiments of the present disclosure, but the protection scope of the present disclosure is not limited thereto. Any changes or substitutions that those skilled in the art can easily think of within the technical scope disclosed by the present disclosure, should be comprised within the protection scope of the present disclosure. Therefore, the protection scope of the present disclosure should be based on the protection scope of the claims.

1. A crosslinking agent, having a general formula R—(R1)ₙ, wherein R comprises at least three reaction sites connected with R1, and R is selected from any one of following groups: 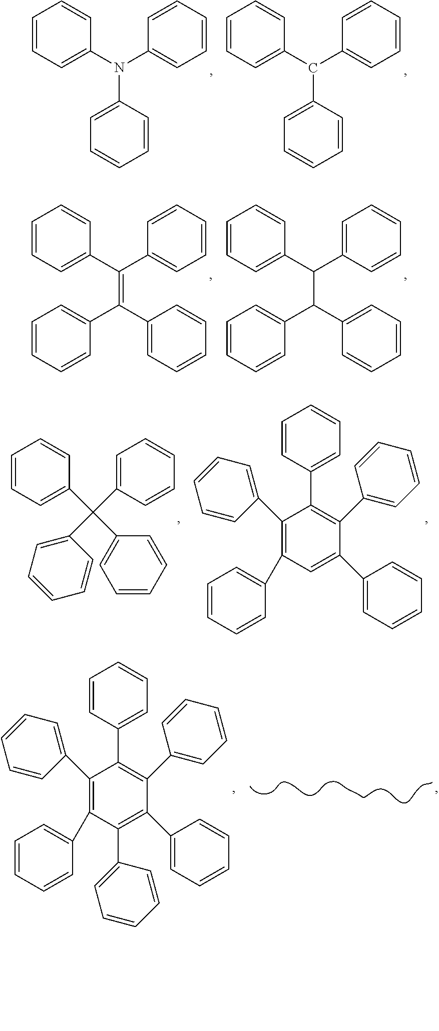
wherein 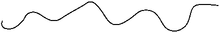
represents a polymer chain, and functional groups of the polymer chain comprise at least one of carboxyl group, hydroxyl group and aldehyde group, and
wherein R1 comprises an azide group N₃, n is a positive integer greater than or equal to 3, and a value of n is equal to a number of the reaction sites.

2. The crosslinking agent according to claim 1, wherein a chemical formula of R1 is 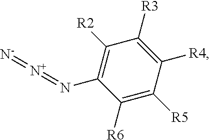
wherein any one of R2~R6 is a conjugated group or a non-conjugated group, and the conjugated group or the non-conjugated group is connected with the reaction site on R, and
wherein remaining groups in R2~R6 except the group connected with the reaction site on R are same as or different from each other, and are respectively selected from fluorine atoms or hydrogen atoms.

3. The crosslinking agent according to claim 2, wherein the non-conjugated group comprises at least one of ester group, amide group, ether group, imine group and carbon-nitrogen double bond, and wherein the conjugated group comprises a carbon-carbon double bond.

4. (canceled)

5. The crosslinking agent according to claim 2, wherein the chemical formula of R1 is 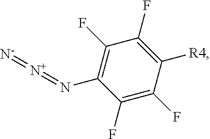
wherein R4 is connected with the reaction site on R.

6. The crosslinking agent according to claim 5, wherein n is equal to 4, and a chemical formula of the crosslinking agent is

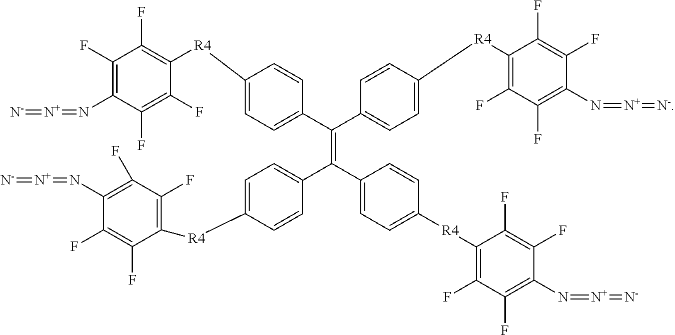

7. The crosslinking agent according to claim 6, wherein the chemical formula of the crosslinking agent is

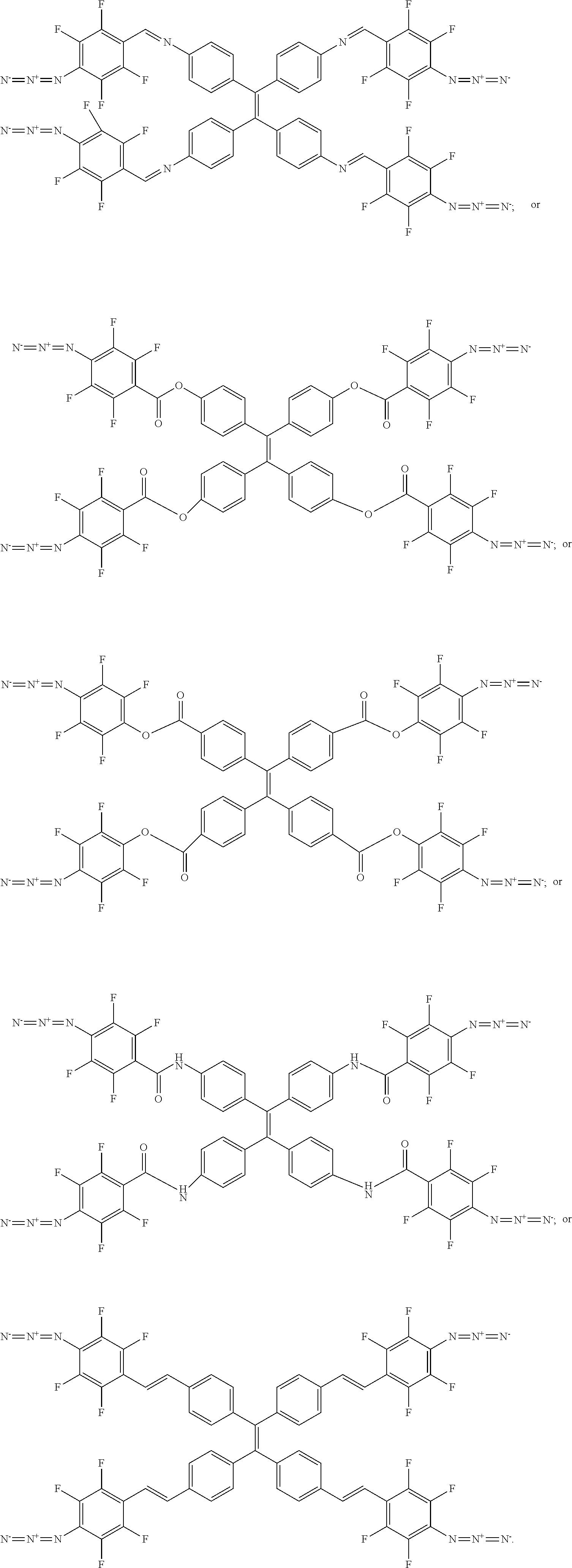

8. A quantum dot, comprising a quantum dot body and a ligand coordinated on the quantum dot body, wherein the ligand comprises a carbon-hydrogen bond, and the ligand is configured to crosslink with the crosslinking agent according to claim 1 under light radiation.

9. The quantum dot according to claim 8, wherein the ligand is selected from any one of oleylamine, isooctyl mercaptan, mercaptoacetic acid, mercaptopropionic acid, oleic acid and 2-mercaptoethyl Boc amine.

10. The quantum dot according to claim 9, wherein the quantum dot is configured to emit red light or blue light, and the ligand is oleic acid, or
wherein the quantum dot is configured to emit green light, and the ligand is 2-mercaptoethyl Boc amine.

11. (canceled)

12. A quantum dot composition, comprising a mixed solution, wherein the mixed solution comprises quantum dots according to claim 8, the crosslinking agent, and a solvent, wherein a concentration of the quantum dots in the mixed solution is about 20~35 mg/ml, and a concentration of the crosslinking agent in the mixed solution is about 0.1~2.0 mg/ml.

13. The quantum dot composition according to claim 12, wherein the solvent is selected from any one of propylene glycol methyl ether acetate, toluene, chlorobenzene, octane, water and alcohol solvents.

14. A quantum dot light emitting layer, comprising a plurality of quantum dots, wherein at least some of the quantum dots comprise quantum dot bodies and ligands coordinated on the quantum dot bodies, and the quantum dot light emitting layer is generated by crosslinking quantum dots with the ligands and the crosslinking agent according to claim 1.

15. The quantum dot light emitting layer according to claim 14, wherein a chemical formula of the quantum dot light emitting layer is R—(R1′-R7-QD)ₙ, wherein QD represents quantum dots, R7 represents the ligand and comprises carbon-hydrogen bonds, and R1′ represents a group in which the azide group N3 of the group R1 is replaced by a group NH.

16. The quantum dot light emitting layer according to claim 15, wherein the chemical formula of the quantum dot light emitting layer is 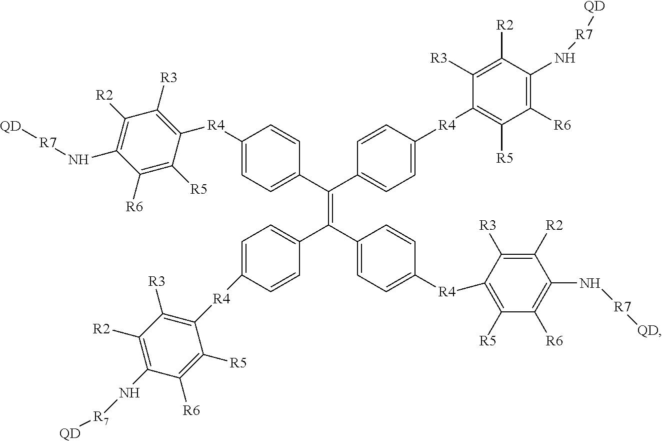
wherein R4 is a conjugated group or a non-conjugated group, and R2, R3, R5 and R6 are same or different from each other and are respectively selected from fluorine atoms or hydrogen atoms.

17. The quantum dot light emitting layer according to claim 16, wherein the quantum dot light emitting layer is configured to emit red light or blue light, the chemical formula of the quantum dot light emitting layer is 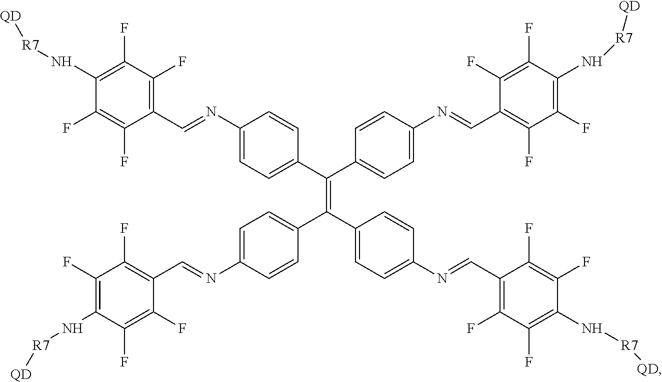
and R7 is oleic acid, or
wherein the quantum dot light emitting layer is configured to emit green light, the chemical formula of the quantum dot light emitting layer is 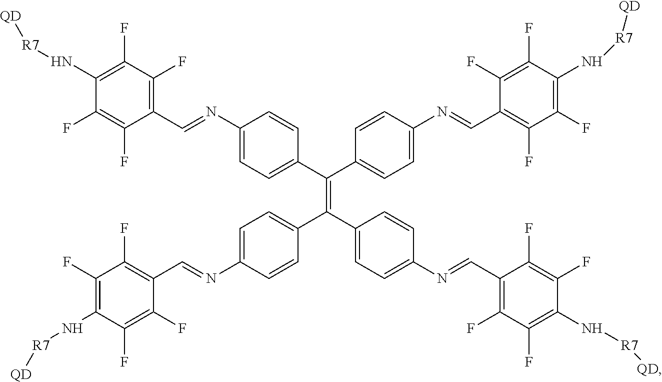
and R7 is 2-mercaptoethyl Boc amine.

18. (canceled)

19. The quantum dot light emitting layer according to claim 14, wherein the quantum dot light emitting layer comprises a plurality of first quantum dot patterns, a plurality of second quantum dot patterns, a plurality of first sub-pixel regions, and a plurality of second sub-pixel regions, the first quantum dot patterns are configured to emit red light and the second quantum dot patterns are configured to emit green light, the first quantum dot patterns are only in the first sub-pixel regions but not in the second sub-pixel regions, and the second quantum dot patterns are only in the second sub-pixel regions but not in the first sub-pixel regions, and
wherein the quantum dot light emitting layer further comprises a plurality of third quantum dot patterns configured to emit blue light and a plurality of third sub-pixel regions, the first quantum dot patterns are only in the first sub-pixel regions but not in the second sub-pixel regions and the third sub-pixel regions, the second quantum dot pattern are only in the second sub-pixel regions but not in the first sub-pixel regions and the third sub-pixel region, and the third quantum dot patterns are only in the third sub-pixel regions but not in the first sub-pixel regions and the second sub-pixel regions.

20. (canceled)

21. A light emitting device, comprising:
a first electrode layer;
an electron transport layer on the first electrode layer;
the quantum dot light emitting layer according to claim 14, on a side of the electron transport layer away from the first electrode layer;
a hole transport layer on a side of the quantum dot light emitting layer away from the first electrode layer; and
a second electrode layer on a side of the hole transport layer away from the first electrode layer.

22. The light emitting device according to claim 21, wherein the electron transport layer is nanoparticles.

23. A display device, comprising a plurality of light emitting devices according to claim 21, wherein at least two of the plurality of light emitting devices are configured to emit light of different colors.

24. A method for preparing a patterned quantum dot light emitting layer, comprising:
providing a substrate;
applying a mixed solution on the substrate, wherein the mixed solution comprises quantum dots and a crosslinking agent, wherein the quantum dots comprise quantum dot bodies and ligands coordinated on the quantum dot bodies, the ligands comprise carbon-hydrogen bonds, and the crosslinking agent is the crosslinking agent according to claim 1;
curing the mixed solution to form a cured layer; and
exposing and developing the cured layer to form a patterned quantum dot light emitting layer.

25. (canceled)
